SEQUENCE LISTING (22 sequences)

SEQ ID NO: 1 - protein, 400 aa, Homo sapiens
  REGION 1..400: note = SEQ ID NO:1
TGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGAD

SEQ ID NO: 2 - protein, 80 aa, Homo sapiens
  REGION 1..80: note = SEQ ID NO:2
GPPGPPGKNGDDGEAGKPGRPGERGPPGPQGARGLPGTAGLPGMKGHRGFSGLDGAKGDAGPAGPKGEPGSPGENGAPGQ

SEQ ID NO: 3 - protein, 300 aa, Homo sapiens
  REGION 1..300: note = SEQ ID NO:3
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQ

SEQ ID NO: 4 - protein, 697 aa, Homo sapiens
  REGION 1..697: note = SEQ ID NO:4
PIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 5 - protein, 75 aa, synthetic construct
  REGION 1..75: note = SEQ ID NO:5
MRFPSIFTAVLFAASSALAAPVNTTTEDETAQIPAEAVIGYSDLEGDFDVAVLPFSASIAAKEEGVSLEKREAEA

SEQ ID NO: 6 - protein, 11 aa, Homo sapiens
  REGION 1..11: note = SEQ ID NO:6
GFSGLDGAKGD

SEQ ID NO: 7 - protein, 475 aa, synthetic construct
  REGION 1..475: note = SEQ ID NO:7
MRFPSIFTAVLFAASSALAAPVNTTTEDETAQIPAEAVIGYSDLEGDFDVAVLPFSASIAAKEEGVSLEKREAEATGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGAD

SEQ ID NO: 8 - protein, 155 aa, synthetic construct
  REGION 1..155: note = SEQ ID NO:8
MRFPSIFTAVLFAASSALAAPVNTTTEDETAQIPAEAVIGYSDLEGDFDVAVLPFSASIAAKEEGVSLEKREAEAGPPGPPGKNGDDGEAGKPGRPGERGPPGPQGARGLPGTAGLPGMKGHRGFSGLDGAKGDAGPAGPKGEPGSPGENGAPGQ

SEQ ID NO: 9 - protein, 866 aa, synthetic construct
  REGION 1..866: note = SEQ ID NO:9
MRFPSIFTAVLFAASSALAAPVNTTTEDETAQIPAEAVIGYSDLEGDFDVAVLPFSASIAAKEEGVSLEKREAEAQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPEKRTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADEKRDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 10 - protein, 546 aa, synthetic construct
  REGION 1..546: note = SEQ ID NO:10
MRFPSIFTAVLFAASSALAAPVNTTTEDETAQIPAEAVIGYSDLEGDFDVAVLPFSASIAAKEEGVSLEKREAEAQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPEKRGPPGPPGKNGDDGEAGKPGRPGERGPPGPQGARGLPGTAGLPGMKGHRGFSGLDGAKGDAGPAGPKGEPGSPGENGAPGQEKRDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 11 - protein, 353 aa, synthetic construct
  REGION 1..353: note = SEQ ID NO:11
MRFPSIFTAVLFAASSALAAPVNTTTEDETAQIPAEAVIGYSDLEGDFDVAVLPFSASIAAKEEGVSLEKREAEAQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQ

SEQ ID NO: 12 - protein, 356 aa, synthetic construct
  REGION 1..356: note = SEQ ID NO:12
MRFPSIFTAVLFAASSALAAPVNTTTEDETAQIPAEAVIGYSDLEGDFDVAVLPFSASIAAKEEGVSLEKREAEAQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPEKRGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQ

SEQ ID NO: 13 - protein, 772 aa, synthetic construct
  REGION 1..772: note = SEQ ID NO:13
MRFPSIFTAVLFAASSALAAPVNTTTEDETAQIPAEAVIGYSDLEGDFDVAVLPFSASIAAKEEGVSLEKREAEAPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 14 - protein, 775 aa, synthetic construct
  REGION 1..775: note = SEQ ID NO:14
MRFPSIFTAVLFAASSALAAPVNTTTEDETAQIPAEAVIGYSDLEGDFDVAVLPFSASIAAKEEGVSLEKREAEAPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRAEKRDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 15 - protein, 493 aa, synthetic construct
  REGION 1..493: note = SEQ ID NO:15
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPEKRGPPGPPGKNGDDGEAGKPGRPGERGPPGPQGARGLPGTAGLPGMKGHRGFSGLDGAKGDAGPAGPKGEPGSPGENGAPGQEKRDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 16 - protein, 300 aa, synthetic construct; identical to SEQ ID NO: 3
  REGION 1..300: note = SEQ ID NO:16

SEQ ID NO: 17 - protein, 719 aa, synthetic construct
  REGION 1..719: note = SEQ ID NO:17
MFSFVDLRLLLLLAATALLTHGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 18 - protein, 534 aa, Homo sapiens
  REGION 1..534: note = SEQ ID NO:18
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLRRATISNPITGDLETVHYRISKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 19 - protein, 491 aa, Homo sapiens
  REGION 1..491: note = SEQ ID NO:19
DAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 20 - protein, 1464 aa, Homo sapiens
  REGION 1..1464: note = SEQ ID NO:20
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 21 - protein, 278 aa, synthetic construct
QEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQ

SEQ ID NO: 22 - protein, 122 aa, synthetic construct
GPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQ

(19) United States
(12) Patent Application Publication
(10) Pub. No.: US 2025/0074969 A1
(43) Pub. Date: Mar. 6, 2025

(54) Novel Recombinant Collagen Polypeptides and Molecules

(71) Applicant: Cambrium GmbH, Berlin (DE)

(72) Inventors: Tamara GERBERT, Berlin (DE); Charles COTTON, Berlin (DE); Pierre SALVY, Berlin (DE); Lucile BONNIN, Berlin (DE)

(21) Appl. No.: 18/728,695

(22) PCT Filed: Jan. 16, 2023
(86) PCT No.: PCT/EP2023/050880

§ 371 Date: Jul. 12, 2024

(30) Foreign Application Priority Data
Jan. 14, 2022 (EP) 22151548.9

(51) Int. Cl. C07K 14/78; A61K 8/65; A61Q 19/08; C12N 9/02; C12N 15/81; C12R 1/84

(52) U.S. Cl. CPC C07K 14/78; A61K 8/65; A61Q 19/08; C12N 9/0071; C12N 15/815; C12R 2001/84; C12Y 114/11002

(57) ABSTRACT

The present invention relates to novel recombinant collagen polypeptides and collagen molecules, polynucleotides encoding the same, methods for preparing them, and compositions and uses thereof. The novel recombinant collagen polypeptides or collagen molecules may be used in cosmetic, personal care, nutraceutical and biomedical applications on account of their multiple benefits for skin, including preventative anti-aging, curative anti-aging, antioxidant, anti-pigmentation, moisturising, anti-wrinkle and elasticity-promoting activities.

### FIELD OF THE INVENTION

The present invention relates to novel recombinant collagen polypeptides and collagen molecules, polynucleotides encoding the same, methods for preparing them, and compositions and uses thereof. The novel recombinant collagen polypeptides or collagen molecules may be used in cosmetic, personal care, nutraceutical and biomedical applications on account of their multiple benefits for skin, including preventative anti-aging, curative anti-aging, antioxidant, anti-pigmentation, moisturising, anti-wrinkle and elasticity-promoting activities.

### BACKGROUND OF THE INVENTION

The cosmetic industry has been focusing on preventing skin aging and skin damage with different chemical and mechanical treatments for several years. Intrinsic skin aging is an inevitable process responsible for skin thinning, drying, and the appearance of wrinkles. On the other hand, extrinsic damage to skin is caused by environmental aggressions such as air pollution or sun exposure. This also results in the appearance of wrinkles, loss of elasticity and the deterioration of skin structure.

Collagen is the main structural protein in human skin. Human skin has an upper layer (the epidermis) and a lower layer (the dermis). Collagen is specifically present in the extracellular matrix (ECM) and is responsible for providing structure and support to surrounding cells present in the epidermis and dermis. Structural changes in the human ECM are known to cause skin aging over time. Mechanisms leading to these changes include both intrinsic and extrinsic factors which affect the molecular and mechanical integrity of the main components of the skin.

Strategies to manage skin aging have therefore been to (1) slow down the intrinsic aging process by nourishing and hydrating the skin properly, (2) protect against extrinsic factors such as UV exposure and skin oxidation by shielding the skin, and (3) compensate for the lack of intrinsic stable collagen with aging.

Skin care products which aim to employ the above strategies often contain collagen. Collagen and gelatin, which is denatured and partially degraded collagen, are isolated from animal tissues. Typical collagen products for the cosmetic market can be classified into full-length or hydrolysed collagen products. Collagen products which use extracted full-length collagen tend to exhibit moisturising effects, but are generally too large to penetrate the skin barrier. Full-length collagen tends to act as a layer which sits on top of the skin. Hydrolysed collagen, i.e. the full length protein degraded into smaller pieces (<1000 Da), is believed to have antimicrobial properties and a higher bioavailability than longer collagen polypeptides, and can be generated by enzymatic digestion or incubation in acid or base. In such a method, many peptides derived from the full-length animal collagen protein are produced, representing a heterogeneous mixture. Some of these peptides are short enough to penetrate the skin more effectively than full-length collagen, but their active concentration will necessarily be low.

However, a need for alternative sources of collagen for skin care products has arisen due to concerns related to cross-species bio-compatibility and immunogenicity of animal-derived collagens, the risk of animal-derived contaminating pathogens and difficulties in the reproducible isolation of collagen preparations with identical chemical compositions. Indeed, variability in gelatin preparations presents a significant problem in their use. Furthermore, many consumers prefer collagen preparations derived from non-animal (i.e., vegan) sources.

Cosmetic products which use alternatively sourced collagen (e.g., bacterial collagen) may use proteins similar in structure to the human protein, but typically do not replicate the actual amino acid composition of the human protein and are therefore not as effective as human collagen.

Accordingly, there exists a need for alternatively sourced collagen, which is not only the same or similar in structure to the human protein, but which compared to prior art collagen molecules provides superior anti-aging and skin healing properties for cosmetic, personal care, nutraceutical and biomedical use. The novel collagen polypeptides and collagen molecules of the present invention fulfil these needs.

### SUMMARY OF THE INVENTION

The invention provides collagen polypeptides comprising sequences derived from human Type I alpha 1 (A1) collagen, and which are capable of being secreted from cultured yeast cells. These collagen polypeptides may also form higher order structures, in particular homotrimeric collagen molecules. Remarkably, the collagen polypeptides and collagen molecules described herein are associated with a diversity of beneficial effects for skin, including preventative anti-aging, curative anti-aging, antioxidant, anti-pigmentation, moisturising, anti-wrinkle and elasticity-promoting activities. The collagen polypeptides and molecules described herein are thus associated with remarkable anti-aging effects.

Accordingly, one aspect of the invention provides a collagen polypeptide comprising an amino acid sequence which is at least 85% identical to SEQ ID NO: 2, wherein the collagen polypeptide has a molecular weight of 1 to 80 kDa.

Another aspect of the invention provides a collagen molecule comprising a homotrimer of three collagen polypeptides as described herein.

Another aspect of the invention provides a nucleic acid encoding a collagen polypeptide described herein, or a polypeptide at least 85% identical thereto, as well as expression vectors comprising the same.

Another aspect of the invention provides a host cell comprising the expression vector or the nucleic acid described herein.

Another aspect of the invention provides a composition comprising the collagen polypeptide or the collagen molecule described herein, and one or more excipients, as well as uses of said composition.

Another aspect of the invention provides a method of preparing a recombinant collagen polypeptide or collagen molecule, comprising the following steps:
(a) providing a nucleotide sequence encoding a collagen polypeptide comprising an amino acid sequence which is at least 85% identical to SEQ ID NO: 2, wherein the encoded collagen polypeptide has a molecular weight of 1-80 kDa;
(b) optionally, providing a nucleotide sequence encoding a human prolyl-4-hydroxylase enzyme,
(c) inserting the nucleotide sequences of (a) and optionally (b) into at least one vector, and
(d) expressing the vector(s) of (c) in cultured yeast cells, preferably Pichia pastoris, and
(e) isolating from the cultured yeast cells a recombinant collagen polypeptide or collagen molecule.

Yet another aspect of the invention provides a recombinant collagen polypeptide or collagen molecule prepared by a method described herein.

### BRIEF DESCRIPTION OF THE FIGURES

FIG. 1: SDS-PAGE gels show the expression and secretion by Pichia pastoris of different collagen polypeptides (produced with constructs #2 (SEQ ID NO: 8), #4 (SEQ ID NO: 10), #6 (SEQ ID NO: 12), and #8 (SEQ ID NO: 14)—see Table 2). The arrows point at the compounds of interest. The asterisks show the P4H subunits.

FIG. 2: Comparison of pepsin digested and non-digested protein suspension by SDS-PAGE 4-16% analysis (for full length constructs #5 (SEQ ID NO: 11) and #6 (SEQ ID NO: 12), showing that homotrimeric collagen molecules are produced and secreted by Pichia pastoris, as the bands indicated by arrows are resistant to digestion by pepsin. The same appears to be true for the cleaved products (SEQ ID NO: 21 and SEQ ID NO: 22) shown as lower weight bands on the gel.

FIG. 3: Shows cytotoxic activity of the tested collagen polypeptides and buffer solution on normal human dermal keratinocytes (panels A-G show the results for Samples 1-7, respectively).

FIG. 4: Shows the biological activity of Samples 2, 4, 5 and 7 on the expression of different genes in NHEK cells. The vertical axis shows the percentage of over- or under-expression compared to cells treated with blank formulation (buffer solutions, see Samples 1, 3 and 6). Samples 2, 4, 5 and 7 are compared to Samples 1, 3, 3 and 6, respectively. MMP1: Matrix metallopeptidase 1; SIRT2/SIRT6: Sirtuin 2 and 6; SOD2: Superoxide dismutase 2 mitochondrial; GLRX: Glutaredoxin; HMOX1: Heme oxygenase 1); AZGP1: Alpha-2-glycoprotein 1, zinc-binding; (−) indicates that the under-expression of this gene may be associated with beneficial effects on the skin. (+) indicates that the over-expression of this gene may be associated with beneficial activity for the skin.

### DETAILED DESCRIPTION OF THE INVENTION

### Definitions

In order for the present invention to be readily understood, several definitions of terms used in the course of the invention are set forth below.

As used herein, the abbreviation “kDa” stands for kilodaltons.

As used herein, the term “amino acid” refers to one of the 20 naturally occurring amino acids or any non-natural analogues. Preferably, the term “amino acid” refers to one of the 20 naturally occurring amino acids.

As used herein, the terms “polypeptide” or “protein” mean a macromolecule composed of a sequence of amino acids. A protein can be a native protein, that is, a protein produced by a naturally-occurring and non-recombinant cell; or it can be produced by a genetically-engineered or recombinant cell, and comprise molecules having the amino acid sequence of the native protein, or molecules having deletions from, additions to, and/or substitutions of one or more amino acids of the native sequence.

The term “sequence identity” indicates a quantitative measure of the degree of homology between two sequences, which can be nucleotide (also termed nucleic acid) sequences or amino acid sequences. If the two sequences to be compared are not of equal length, they must be aligned to give the best possible fit, allowing the insertion of gaps or alternatively, truncation at the ends of the nucleic acid sequences or amino acid sequences. The skilled person will acknowledge that various means for comparing sequence identity are available (see below).

As used herein, a “conservative amino acid substitution” means that the amino acid can be substituted by another amino acid in its respective group, according to the following six groups: [1] Alanine (A), Serine (S), Threonine (T); [2] Aspartic acid (D), Glutamic acid (E); [3] Asparagine (N), Glutamine (Q); [4] Arginine (R), Lysine (K); [5] Isoleucine (I), Leucine (L), Methionine (M), Valine (V); and [6] Phenylalanine (F), Tyrosine (Y), Tryptophan (W).

As used herein, the term “homotrimer” means a complex of three collagen polypeptides of the same type, i.e., three human Type I A1 collagen polypeptides.

As used herein, “recombinant” refers to a polypeptide or protein molecule which is made using recombinant techniques, i.e., which is not naturally occurring. Methods and techniques for the production of recombinant nucleic acids and polypeptides are well known in the art.

As used herein, “isolated” refers to a protein that is removed from cell culture and separated from cell culture components, e.g., it may have been separated from at least 90% of cell culture components.

As used herein, a molecule that is an “antioxidant” has antioxidant activity, i.e., it is capable of stimulating native antioxidant pathways, e.g., in skin cells.

As used herein, a molecule that has “anti-aging activity” is capable of slowing down intrinsic aging processes in skin cells, e.g., in normal human dermal keratinocytes, by differential transcriptional regulation of genes involved in aging processes (compared to baseline expression levels), including, for example, genes described to be involved in preventive and curative anti-aging, antioxidant and anti-pigmentation (as determined, for example, via mRNA sequencing).

A “vector” is a nucleic acid that can be used to introduce another nucleic acid (or “construct”) linked to it into a cell, e.g., by electroporation. One type of vector is a “plasmid”, which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) integrate into the genome of a host cell upon introduction into the host cell and culturing under selective pressure, and thereby are replicated along with the host genome. A vector can be used to direct the expression of a chosen nucleic acid in a cell.

A “host cell” is a cell that can be used to express a nucleic acid, e.g., a nucleic acid disclosed herein. A host cell in accordance with the invention is a yeast cell, preferably Pichia pastoris.

As used herein, a “subject” includes all mammals, preferably humans.

As used herein, a “patient” refers to a subject to be treated.

As used herein, “treatment” is synonymous with reducing symptoms of a disease or a condition, inhibiting progression of the disease or condition, causing regression of the disease or condition and/or curing the disease or condition. The term “treatment” in the present invention is meant to include therapeutic treatment as well as prophylactic or suppressive measures for a disease or condition.

The terms “of the invention” or “according to the invention” as used herein are intended to refer to all aspects and embodiments of the invention disclosed and/or claimed herein. Any aspects, items or embodiments referred to herein as being “disclosed herein” or “described herein” are to be understood as being aspects, items or embodiments “of the invention” or “according to the invention”.

As used herein, the term “comprising” is to be construed as encompassing both “including” and “consisting of”, both meanings being specifically intended, and hence individually disclosed, embodiments according to the present invention.

As used herein, the articles “a” and “an” preceding an element or component are intended to be nonrestrictive regarding the number of instances (i.e., occurrences) of the element or component.

Therefore, “a” or “an” is to be read to include one or at least one, and the singular word form of the element or component also includes the plural unless the number is obviously meant to be singular.

### Collagen Polypeptides

In one aspect, the invention provides a collagen polypeptide comprising an amino acid sequence which is at least 85% identical to SEQ ID NO: 2, wherein the collagen polypeptide has a molecular weight of 1 to 80 kDa. The amino acid sequence of human Type I A1 collagen is provided in SEQ ID NO: 20 herein.

In some embodiments, the collagen polypeptide has a molecular weight of 2 to 45 kDa. In some embodiments, the collagen polypeptide has a molecular weight of 5 to 20 kDa. It is believed that the size of the collagen polypeptides of the invention allows them to penetrate the skin more effectively than full-length collagen.

In some embodiments, the collagen polypeptide is a fragment of human Type I A1 collagen.

In some embodiments, the collagen polypeptide may comprise at least two PGP amino acid sequences.

In some embodiments, the amino acid sequence of the collagen polypeptide comprises at least 10% proline. Preferably, the amino acid sequence of the collagen polypeptide comprises at least 15% proline. More preferably, the amino acid sequence of the collagen polypeptide comprises at least 20% proline.

In some embodiments, the amino acid sequence of the collagen polypeptide comprises at least 1.5%, at least 2.5%, at least 5% or at least 7.5% hydroxyproline. A higher hydroxyproline ratio in the collagen is expected to correspond to a higher moisturization and anti-aging activity. A higher hydroxyproline ratio is also expected to improve performance as a nutraceutical, as one potential mechanism of action involves the passage of hydroxyproline containing dipeptides and tripeptides into the bloodstream.

In some embodiments, the amino acid sequence of the collagen polypeptide comprises at least 15% glycine. Preferably, the amino acid sequence of the collagen polypeptide comprises at least 20% glycine. More preferably, the amino acid sequence of the collagen polypeptide comprises at least 25% glycine.

In some embodiments, the amino acid sequence of the collagen polypeptide comprises hydroxylated lysines.

It is believed that the proline/hydroxyproline, glycine and/or hydroxylated lysine-rich amino acid sequence of the collagen polypeptides described herein contributes to their superior moisturizing and anti-aging effects on human skin.

In some embodiments, the collagen polypeptide has an isoelectric point of >7, preferably >8.

In some embodiments, the collagen polypeptide is an antioxidant.

For example, the collagen polypeptide may preferably comprise an amino acid sequence which is GFSGLDGAKGD (SEQ ID NO: 6) or which differs from GFSGLDGAKGD (SEQ ID NO: 6) by up to three conservative amino acid substitutions.

In some embodiments, the collagen polypeptide has anti-aging activity. Thus, in certain embodiments, products containing the collagen polypeptide of the invention will slow down intrinsic aging processes in skin cells by differential transcriptional regulation of genes involved in aging processes.

Accordingly, in some embodiments, the collagen polypeptide downregulates expression of MMP1 in skin cells by at least 20%, preferably by at least 40%, more preferably by at least 60% (as compared to baseline expression level). In other embodiments, the collagen polypeptide upregulates expression of SIRT2/SIRT6 in skin cells by at least 10%, preferably by at least 30%, more preferably by at least 50% (as compared to baseline expression level). In other embodiments, the collagen polypeptide upregulates expression of SOD2 in skin cells by at least 30%, preferably by at least 60%, more preferably by at least 90% (as compared to baseline expression level). In some embodiments, the collagen polypeptide upregulates expression of GLRX in skin cells by at least 20%, preferably by at least 40%, more preferably by at least 60% (as compared to baseline expression level). In other embodiments, the collagen polypeptide upregulates expression of HMOX1 in skin cells by at least 40%, preferably by at least 80%, more preferably by at least 120%, most preferably by at least 150% (as compared to baseline expression level). In other embodiments, the collagen polypeptide upregulates expression of AZGP1 in skin cells by at least 30%, preferably by at least 60%, more preferably by at least 90% (as compared to baseline expression level).

In preferred embodiments, the collagen polypeptide upregulates the expression of one or more of SIRT2/SIRT6, SOD2, GLRX, HMOX1 and AZGP1 in skin cells (as compared to baseline expression levels). In some particularly preferred embodiments, the collagen polypeptide upregulates the expression of SIRT2/SIRT6, SOD2, GLRX, HMOX1 and AZGP1 in skin cells (as compared to baseline expression levels). In some of these embodiments, the collagen polypeptide downregulates expression of MMP1 in skin cells (as compared to baseline expression level).

In some preferred embodiments, the collagen polypeptide upregulates the expression of one or more of SIRT2/SIRT6, SOD2, GLRX, HMOX1 and AZGP1 in skin cells by at least 50% (as compared to baseline expression levels) and downregulates expression of MMP1 in skin cells by at least 50% (as compared to baseline expression level).

In some embodiments, the collagen polypeptide is hypoallergenic. Thus, in certain embodiments, products containing the collagen polypeptide of the invention will not elicit an allergic reaction in a subject when applied to said subject.

In some embodiments, the collagen polypeptide comprises an amino acid sequence which is at least 85% identical to, at least 90% identical to, at least 95% identical to, or at least 98% identical to, any one of SEQ ID NOs: 1 to 4 or 7 to 17. Preferably, the collagen polypeptide comprises an amino acid sequence which is selected from SEQ ID NOs: 1 to 4 and 7 to 17.

In preferred embodiments, the collagen polypeptide comprises an amino acid sequence which is at least 90% identical to, at least 95% identical to, or at least 98% identical to, SEQ ID NO: 2. In particularly preferred embodiments, the collagen polypeptide comprises an amino acid sequence which is SEQ ID NO: 2.

In other preferred embodiments, the collagen polypeptide comprises an amino acid sequence which is at least 85% identical to, at least 90% identical to, at least 95% identical to, or at least 98% identical to any one of SEQ ID NOs: 3, 21 and 22. In some of these embodiments, the collagen polypeptide comprises the amino acid sequence of any one of SEQ ID NOs: 3, 21 and 22.

In particular embodiments, the collagen polypeptide comprises an amino acid sequence which is at least 85% identical to, at least 90% identical to, at least 95% identical to, or at least 98% identical to, SEQ ID NO: 8. In particularly preferred embodiments, the collagen polypeptide comprises an amino acid sequence which is SEQ ID NO: 8.

In some particularly preferred embodiments, the collagen polypeptide comprises the amino acid sequence of SEQ ID NO: 2 and has a molecular weight of 5 to 20 kDa. In some of these embodiments, the collagen polypeptide has an isoelectric point of >8. In some of these embodiments, the collagen polypeptide comprises at least 2.5% hydroxyproline.

Protein and/or nucleic acid sequence identities (homologies) can be evaluated using any of the variety of sequence comparison algorithms and programs known in the art. For sequence comparison, typically one sequence acts as a reference sequence (e.g., a sequence disclosed herein), to which test sequences are compared. A sequence comparison algorithm then calculates the percent sequence identities for the test sequences relative to the reference sequence, based on the program parameters.

The percent identity of two amino acid or two nucleic acid sequences can be determined for example by the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. Alternatively, the percent identity of two amino acid or two nucleic acid sequences can be determined for example by the DIAMOND algorithm, described in: Benjamin Buchfink, Chao Xie & Daniel H. Huson, Fast and Sensitive Protein Alignment using DIAMOND, Nature Methods, 12, 59-60 (2015).

In some embodiments, the collagen polypeptide is a recombinant collagen polypeptide.

In some embodiments, the collagen polypeptide is an isolated collagen polypeptide.

### Nucleic Acids, Vectors and Host Cells

In another aspect, the invention provides a nucleic acid encoding the collagen polypeptides described herein with all of their embodiments.

Accordingly, the nucleic acid of the invention encodes a polypeptide comprising an amino acid sequence of SEQ ID NO: 2, or a polypeptide at least 85% identical thereto, wherein the collagen polypeptide has a molecular weight of 1 to 80 kDa. In some embodiments, the encoded collagen polypeptide has a molecular weight of 2 to 45 kDa. In some preferred embodiments, the encoded collagen polypeptide has a molecular weight of 5 to 20 kDa.

In some embodiments, the nucleic acid encodes a polypeptide comprising an amino acid sequence of any one of SEQ ID NOs: 3, 21 and 22, or a polypeptide which is at least 85%, at least 90%, at least 95%, or at least 98% identical thereto. Preferably, the nucleic acid encodes a polypeptide comprising the amino acid sequence of any one of SEQ ID NOs: 3, 21 and 22.

In other embodiments, the nucleic acid encodes a polypeptide comprising an amino acid sequence of any one of SEQ ID NOs: 8, 10, 11, 12, 15 and 16, or a polypeptide which is at least 85%, at least 90%, at least 95%, or at least 98% identical thereto. Preferably, the nucleic acid encodes a polypeptide comprising the amino acid sequence of any one of SEQ ID NOs: 8, 10, 11, 12, 15 and 16.

In preferred embodiments, the nucleic acid encodes a polypeptide comprising an amino acid sequence of SEQ ID NO: 8, or a polypeptide which is at least 85%, at least 90%, at least 95%, or at least 98% identical thereto. Preferably, the nucleic acid encodes a polypeptide comprising the amino acid sequence of SEQ ID NO: 8.

In another aspect, the invention provides expression vectors comprising the nucleic acids described herein with all of their embodiments.

In yet another aspect, the invention provides a host cell comprising the expression vector or the nucleic acid described herein with all of their embodiments.

### Collagen Molecules

In another aspect, the invention provides a collagen molecule comprising a homotrimer of three collagen polypeptides as described herein with all of their embodiments.

Thus, as a homotrimer, the collagen molecule of the invention differs from natural human Type I collagen, which is a heterotrimer consisting of two alpha 1 polypeptides and one alpha 2 polypeptide.

In some embodiments, the collagen polypeptide or collagen molecule is glycosylated.

Preferably, the collagen polypeptide or collagen molecule has been secreted from cultured yeast cells, such as Pichia pastoris. In some embodiments, the cultured yeast cells are the wild-type strain. Alternatively, the cultured yeast cells may have a methanol oxidase knocked out of their genome, which results in them growing more slowly on methanol. In preferred embodiments, the cultured yeast cells have a deletion of the AOX1 gene. In some embodiments, the cultured yeast cells stably express prolyl-4-hydroxylase.

It will be understood by the person skilled in the art that collagen polypeptides or collagen molecules produced by recombinant techniques in yeast cells can be classed as vegan, i.e., they are not sourced from an animal or animal product. Thus, in some embodiments, the collagen polypeptides and collagen molecules described herein are vegan.

### Compositions and Uses Thereof

In another aspect, the invention provides a composition comprising the collagen polypeptide as described herein with all of its embodiments or the collagen molecule described herein with all of its embodiments, and one or more excipients.

Excipients can be any excipient known to the skilled person such as for example starch, glucose, lactose, sucrose, silica gel, sodium stearate, glycerol, glycerol monostearate, talc, sodium chloride, propylene, glycol, and ethanol; as well as combinations thereof.

The compositions described herein may comprise a continuous aqueous phase comprising an aqueous carrier and a thickening agent. The aqueous phase may further comprise other hydrophilic substances which exhibit limited solubility in an oil phase, including but not limited to water-soluble ingredients, water-soluble sunscreens and other water-soluble skin care actives. The compositions described herein comprise from about 60% to about 98%, preferably from about 65% to about 97% of an aqueous phase.

The compositions described herein may further comprise components such as carriers, stabilizers, preservatives, thickening agents, emulsifiers, humectants, emollients and/or other components.

Carriers can be any carrier known to the skilled person such as for example aqueous liquids; dextrose solutions; glycerol solutions; microemulsions; nanoparticles; liposomal suspensions; oils, including those of vegetable or synthetic origin, such as peanut oil, soybean oil, mineral oil, and sesame oil; isopropyl alcohol, gaseous fluorocarbons, ethyl alcohol, polyvinyl pyrrolidone, propylene glycol, a gel-producing material, stearyl alcohol, stearic acid, sorbitan monooleate, and methylcellulose; as well as combinations thereof.

Carriers particularly useful in the present invention include water and water solutions of lower alkyl alcohols. Lower alkyl alcohols useful herein are monohydric alcohols having 1 to 6 carbons, more preferably ethanol and glycerol. Preferably, the aqueous carrier is substantially water. Deionized water is preferably used. Water from natural sources including mineral cations can also be used, depending on the desired characteristic of the product.

The pH of the compositions described herein is preferably from about 3 to about 8. The pH may be adjusted to that which provides optimum efficacy of the collagen. Buffers and other pH adjusting agents can be included to achieve the desirable pH. Suitable pH adjusters herein include acetates, phosphates, citrates, sodium hydroxide, triethanolamines, aminomethylpropanol and carbonates.

Stabilizers can be any stabilizers known to the skilled person such as for example amino acids; ascorbic acid; surfactants such as poloxamer; polyhydric sugar alcohols such as glycerin, erythritol, arabitol, xylitol, sorbitol and mannitol; as well as combinations thereof.

The aqueous phase of the compositions described herein comprises from about 0.1% to about 2%, preferably from about 0.2% to about 1.5% of thickening agents, including thickeners, gelling agents, and structuring agents. The level and species of the thickening agent are selected according to the compatibility with other components, and other desired characteristic of the product. Thickening agents include cross-linked polyacrylate polymers and copolymers, hydrophobically-modified polyacrylate polymers and copolymers, polyacrylamide polymers and copolymers, polyacryloyldimethyltaurates, aminomethylpropanol (AMP)-based polymers and copolymers, polysaccharides and gums. Useful herein are carboxylic acid/carboxylate copolymers. Non-limiting examples of carboxylic acid/carboxylate copolymers useful herein include: CTFA name Acrylates/C 10-30 Alkyl Acrylate Crosspolymer having tradenames Pemulen TR-1, Pemulen TR-2, Carbopol Ultrez 10, Carbopol Ultrez 20, Carbopol Ultrez 21, Carbopol 1342, Carbopol 1382, and Carbopol ETD 2020 (all from Noveon), Xanthan gum. Commercially available additional water soluble polymers highly useful herein include xanthan gum with tradename KELTROL series available from Kelco; Carbomers with tradenames Carbopol 934, Carbopol 940, Carbopol950, Carbopol 980 and Carbopol 981 (all from Noveon); acrylates/steareth-20 methacrylate copolymer with tradename ACRYSOL 22 (from Rohm and Hass); polyacrylamide with tradename SEPIGEL 305 (from Seppic); glycerylpolymethacrylate with tradename LUBRAGEL NP, and a mixture of glyceryl polymethacrylate, propylene glycol and PVM/MA copolymer with tradename LUBRAGEL OIL (all from ISP); scleroglucan with tradename Clearogel SC11 (from Michel Mercier Products Inc); ethylene oxide and/or propylene oxide based polymers with tradenames CARBOWAXPEGs, POLYOX WASRs, and UCON FLUIDS (all from Amerchol). Another class of thickening agents includes a structuring agent, such as stearyl alcohol, cetyl alcohol, and behenyl alcohol.

The compositions described herein may contain an emulsifier, useful for dispersing and suspending the oil phases within the aqueous phase. When the compositions described herein contain an emulsifier, they contain an emulsifier no more than 1%, preferably no more than 0.5%, and more preferably no more than 0.2%. Examples of emulsifiers include: polyethylene glycol 20 sorbitan monolaurate(polysorbate 20), steareth-20, ceteareth-20, PPG-2 methyl glucose ether distearate, ceteth-10, polysorbate 80, cetylphosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ethersodium stearate, polyglyceryl-4 isostearate, hexyl laurate, PPG-2 methyl glucose ether distearate, ceteth-10, diethanolaminecetyl phosphate, glyceryl stearate, PEG 40 hydrogenated castor oil, PEG-60 hydrogenated castor oil, Glycereth-25 PCA Isostearate, and mixtures thereof.

Preservatives can be any preservative known to the skilled person in the art, such as for example octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol, diols such as propanediol, butylene glycol and penthylene glycol. Examples of emulsifiers include: Triethyl acetate, Glyceryl caprylate, Benzoic acid, Sorbitan caprylate, Glycerin, Sodium levulinate, Sodium anisate, Penthylene glycol, Phenylpropanol, Gluconolactone, Lactobacillus ferment, Lactobacillus, Cocos Nucifera (Coconut) Fruit Extracts, Fruits extracts, Leuconostoc/Radish Root Ferment Filtrate, Benzyl Alcohol, Dehydroacetic Acid, Potassium sorbate, Sorbic acid, and mixtures thereof.

The compositions described herein may contain one or more additional cosmetic active ingredients useful for enhancing the efficacy of the final cosmetic product. When the compositions described herein contain additional active ingredients, they contain the additional active ingredients no more than 20%, preferably no more than 10%, preferably no more than 1% and more preferably no more than 0.1%. Examples of active ingredients include but is not limited to: hyaluronic acid, hydrolyzed collagen, animal derived collagen, marine collagen, copper peptide, peptides and derivatives, retinoids and derivatives, niacinamide, vitamin C, vitamin E, vitamin K, alpha hydroxy acids (AHAs), beta hydroxy acids (BHAs), ferulic acid, ceramides, and mixtures thereof.

The compositions described herein may further comprise humectants, emollients, exfoliants, non-vitamin antioxidants and radical scavengers, hair growth regulators, minerals, preservatives, phytosterols and/or plant hormones, protease inhibitors, tyrosinase inhibitors, anti-inflammatory agents and N-acyl amino acid compounds. Suitable humectants include, but are not limited to, polyhydric alcohols such as polyalkylene glycols and their derivatives. Illustrative are propylene glycol, dipropylene glycol, polypropylene glycol, polyethylene glycol, sorbitol, hydroxypropylsorbitol, hexylene glycol, 1,3-butylene glycol, 1,2,6-hexanetriol, ethoxylated glycerin, propoxylated glycerinand mixtures thereof. Suitable emollients include, but are not limited to, hydrocarbons, fatty acids, fatty alcohols and esters.

Yet other components in the compositions described herein can be hydrophilic polymers such as polyethylene glycol (PEG); monosaccharides; disaccharides; including mannose and trehalose; oligosaccharides, polysaccharides, and other carbohydrates including dextrins or dextrans; chelating agents such as EDTA; salt-forming counter-ions such as sodium; metal complexes (e.g. Zn-protein complexes); and fatty acid esters, fatty acid ethers or sugar esters.

The compositions described herein can be formulated in a variety of different forms, such as liquid, semi-solid, or solid dosage forms depending on the intended use. In one embodiment, the compositions described herein are formulated in a cream, emulsion, serum, aqueous solution, ointment, paste, lotion, suspension, gel, mask, skin cleaner, such as soap, cleansing cream, cleansing lotion, cleansing milk, cleansing pad, facial wash, hair shampoo, hair conditioner, or body shampoo. Preferably, the composition is comprised in a cream. In another embodiment, the compositions described herein are formulated as solid dosage forms, such as tablets, capsules, granules or powders.

The compositions described herein are intended for external (i.e., as cosmetic or personal care products) use on a subject. The compositions described herein may also be used internally (i.e., as nutraceutical or biomedical products). Accordingly, in some embodiments, the compositions described herein are applied topically, e.g., superficially (e.g., via microneedling) or via deep injection. In other embodiments, the compositions described herein are applied internally.

The compositions described herein compensate for the loss of collagen associated with aging and skin damage.

Thus, the compositions described herein with all of their embodiments may be used in repairing damaged skin, or protecting skin from oxidative damage, pigmentation or aging.

Likewise provided herein is a method of repairing damaged skin, or protecting skin from oxidative damage, pigmentation or aging, comprising administering a composition described herein with all of its embodiments to the skin or skin cells of a subject. Alternatively, the composition may be administered internally.

Further provided herein is the use of a composition described herein with all of its embodiments as a skin moisturizer.

The compositions described herein nourish the skin and are expected to improve skin texture, skin balancing, skin plumping, skin smoothness, and reduce wrinkles.

In some embodiments, the compositions described herein are for use in treating a skin disorder. Examples of skin disorders include acne, rosacea, psoriasis, atopic dermatitis (eczema), contact dermatitis, and vitiligo.

### Methods of Preparing Recombinant Collagen Polypeptides or Collagen Molecules

In another aspect, the invention provides a method of preparing a recombinant collagen polypeptide or collagen molecule, comprising the following steps:
(a) providing a nucleotide sequence encoding a collagen polypeptide comprising an amino acid sequence which is at least 85% identical to SEQ ID NO: 2, wherein the encoded collagen polypeptide has a molecular weight of 1-80 kDa;
(b) optionally, providing a nucleotide sequence encoding a human prolyl-4-hydroxylase enzyme,
(c) inserting the nucleotide sequences of (a) and optionally (b) into at least one vector, and
(d) expressing the vector(s) of (c) in cultured yeast cells, preferably Pichia pastoris, and
(e) isolating from the cultured yeast cells a recombinant collagen polypeptide or collagen molecule.

In some embodiments, the encoded collagen polypeptide has a molecular weight of 2 to 45 kDa. In some embodiments, the encoded collagen polypeptide has a molecular weight of 5 to 20 kDa.

In some embodiments, the encoded collagen polypeptide may comprise at least two PGP amino acid sequences.

In some embodiments, the amino acid sequence of the encoded collagen polypeptide comprises at least 10% proline. Preferably, the amino acid sequence of the encoded collagen polypeptide comprises at least 15% proline. More preferably, the amino acid sequence of the encoded collagen polypeptide comprises at least 20% proline.

In some embodiments, the amino acid sequence of the collagen polypeptide comprises at least 1.5%, at least 2.5%, at least 5% or at least 7.5% hydroxyproline.

In some embodiments, the amino acid sequence of the encoded collagen polypeptide comprises at least 15% glycine. Preferably, the amino acid sequence of the encoded collagen polypeptide comprises at least 20% glycine. More preferably, the amino acid sequence of the encoded collagen polypeptide comprises at least 25% glycine.

In some embodiments, the amino acid sequence of the encoded collagen polypeptide comprises hydroxylated lysines.

In some embodiments, the amino acid sequence of the encoded collagen polypeptide has an isoelectric point of >7, preferably >8.

In some embodiments, the encoded collagen polypeptide is an antioxidant.

For example, the encoded collagen polypeptide may comprise an amino acid sequence which is GFSGLDGAKGD (SEQ ID NO: 6) or which differs from GFSGLDGAKGD (SEQ ID NO: 6) by up to three conservative amino acid substitutions.

In some embodiments, the encoded collagen polypeptide has anti-aging activity.

In some embodiments, the encoded collagen polypeptide is hypoallergenic.

In some embodiments, the encoded collagen polypeptide comprises an amino acid sequence which is at least 85% identical to, at least 90% identical to, at least 95% identical to, or at least 98% identical to, any one of SEQ ID NOs: 1 to 4 or 7 to 17. Preferably, the encoded collagen polypeptide comprises an amino acid sequence which is selected from SEQ ID NOs: 1 to 4 and 7 to 17.

In preferred embodiments, the encoded collagen polypeptide comprises an amino acid sequence which is at least 90% identical to, at least 95% identical to, or at least 98% identical to, SEQ ID NO: 2. In particularly preferred embodiments, the encoded collagen polypeptide comprises an amino acid sequence which is SEQ ID NO: 2.

In other preferred embodiments, the encoded collagen polypeptide comprises an amino acid sequence which is at least 85% identical to, at least 90% identical to, at least 95% identical to, or at least 98% identical to, any one of SEQ ID NOs: 3, 21 and 22. In some of these embodiments, the encoded collagen polypeptide comprises the amino acid sequence of any one of SEQ ID NOs: 3, 21 and 22.

In some embodiments, the encoded collagen polypeptide comprises an amino acid sequence which is at least 85% identical to, at least 90% identical to, at least 95% identical to, or at least 98% identical to, any one of SEQ ID NOs: 8, 10, 11, 12, 15 and 16. Preferably, the encoded collagen polypeptide comprises an amino acid sequence which is selected from any one of SEQ ID NOs: 8, 10, 11, 12, 15 and 16.

In preferred embodiments, the encoded collagen polypeptide comprises an amino acid sequence which is at least 85% identical to, at least 90% identical to, at least 95% identical to, or at least 98% identical to SEQ ID NO: 8. In some of these embodiments, the encoded collagen polypeptide comprises an amino acid sequence which is SEQ ID NO: 8.

Various means for comparing sequence identity are disclosed hereinabove.

In some embodiments, the recombinant collagen molecule produced by the method comprises a homotrimer of three collagen polypeptides as described herein with all of their embodiments.

In some embodiments, the recombinant collagen polypeptide or collagen molecule produced by the method is glycosylated.

In some embodiments, the nucleotide sequence encoding a collagen polypeptide which is a fragment of human Type I A1 collagen is preceded by a nucleotide sequence encoding a secretion signal sequence. For example, the nucleotide sequence encoding a secretion signal sequence may encode the MatAlphaD secretion signal sequence (amino acid sequence of SEQ ID NO: 5). Other secretion signal sequences may also be used, such as OST1, MatAlphaF, Invertase, and the native secretion signal from Homo sapiens.

In some embodiments, the human prolyl-4-hydroxylase enzyme is encoded by amino acid sequences according to SEQ ID NO: 18 and SEQ ID NO: 19.

In some embodiments, the nucleotide sequences of (a) and/or (b) are inserted into a vector which uses a bi-directional promoter.

In some embodiments, the nucleotide sequences of (a) and/or (b) are inserted into a vector which uses an inducible and de-repressible promoter. For example, the promoter may be a methanol-inducible promoter.

In some embodiments, the nucleotide sequences of (a) and/or (b) are inserted into a vector which allows post-translational translocation into the endoplasmic reticulum.

The vectors can be introduced into yeast cells by conventional means, such as transfection, transduction or electroporation. Preferably, the vectors are introduced into yeast cells by electroporation of linearized vectors.

Yeast cells can be cultured at about 17 to about 23° C. (e.g., about 20° C.), with sufficient nutrients such as a carbon source (e.g., glucose), a nitrogen source (e.g., NH4+), salts (e.g., Na⁺, K⁺, Mg²⁺, Ca²⁺, etc.), trace elements, peptone (a water-soluble mixture of polypeptides and amino acids formed by the partial hydrolysis of protein), and cultivation at a suitable pH (e.g., an acidic pH). Sufficient nutrients can be provided by yeast growth medium as known in the art, e.g., yeast and mold “YM” medium or yeast extract-peptone-dextrose (YPD) medium. Typically, a yeast growth medium is a selective growth medium of acidic pH which permits the growth of yeast, while deterring growth of bacteria and other acid-intolerant organisms.

In some embodiments, the yeast cells used in the method are the wild-type strain. Alternatively, the yeast cells used in the method may have a methanol oxidase knocked out of their genome, which results in them growing more slowly on methanol. In preferred embodiments, the yeast strain used in the method has a deletion of the AOX1 gene. An example of such yeast is the MutS strain, which is commercially available from Invitrogen (KM71H), Graz University of Technology (CBS7435MutS) or Biogrammatics (BG11). MutS strains still express Aox2 but grow slower than wild type strains when methanol is used as the sole carbon source.

In some embodiments, the recombinant collagen polypeptide or collagen molecule is isolated from the culture supernatant of the cultured yeast cells. For example, centrifugation, precipitation or filtration may be used in this regard.

In some embodiments, the recombinant collagen molecule is an antioxidant.

In some embodiments, the recombinant collagen polypeptide or collagen molecule produced by the method described herein is vegan, i.e., the method does not employ animal products.

Also provided herein is a recombinant collagen polypeptide or collagen molecule prepared by the method described herein with all of its embodiments. Said collagen molecule comprises a homotrimer of three collagen polypeptides.

In some embodiments, the recombinant collagen polypeptide prepared by the method described herein comprises at least 1.5%, at least 2.5%, at least 5% or at least 7.5% hydroxyproline.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described herein, what is conceptually equivalent, what can be obviously substituted, and also what incorporates the essential ideas.

### Items

1. A collagen polypeptide, comprising an amino acid sequence which is at least 85% identical to SEQ ID NO: 2, wherein the collagen polypeptide has a molecular weight of 1 to 80 kDa.
2. The collagen polypeptide of item 1, wherein the collagen polypeptide has a molecular weight of 2 to 45 kDa.
3. The collagen polypeptide of item 1, wherein the collagen polypeptide has a molecular weight of 5 to 20 kDa.
4. The collagen polypeptide of any one of items 1-3, wherein the collagen polypeptide is a fragment human Type I A1 collagen.
5. The collagen polypeptide of any one of items 1-4, wherein the collagen polypeptide comprises at least two PGP amino acid sequences.
6. The collagen polypeptide of any one of items 1-5, wherein the amino acid sequence of the collagen polypeptide comprises at least 2.5% hydroxyproline.
7. The collagen polypeptide of any one of items 1-6, wherein the amino acid sequence of the collagen polypeptide comprises at least 15% glycine.
8. The collagen polypeptide of any one of items 1-7, wherein the collagen polypeptide comprises hydroxylated lysines.
9. The collagen polypeptide of any one of items 1-8, wherein the collagen polypeptide has an isoelectric point of >7, preferably >8.
10. The collagen polypeptide of any one of items 1-9, wherein the collagen polypeptide is an antioxidant.
11. The collagen polypeptide of any one of items 1-10, wherein the collagen polypeptide has anti-aging activity.
12. The collagen polypeptide of any one of items 1-11, wherein the collagen polypeptide upregulates the expression of one or more of SIRT2/SIRT6, SOD2, GLRX, HMOX1 and AZGP1 in skin cells by at least 50%, as compared to baseline expression levels.
13. The collagen polypeptide of any one of items 1-12, wherein the collagen polypeptide downregulates expression of MMP1 in skin cells by at least 50%, as compared to baseline expression level.
14. The collagen polypeptide of any one of items 1-13, wherein the collagen polypeptide comprises an amino acid sequence which is GFSGLDGAKGD (SEQ ID NO: 6) or which differs from GFSGLDGAKGD (SEQ ID NO: 6) by up to three conservative amino acid substitutions.
15. The collagen polypeptide of any one of items 1-14, wherein the collagen polypeptide is hypoallergenic.
16. The collagen polypeptide of any one of items 1-15, wherein the collagen polypeptide comprises an amino acid sequence which is at least 85% identical to any one of SEQ ID NOs: 1 to 4 or 7 to 17.
17. The collagen polypeptide of any one of items 1-16, wherein the collagen polypeptide comprises an amino acid sequence which is selected from SEQ ID NOs: 1 to 4 and 7 to 17.
18. The collagen polypeptide of any one of items 1-17, wherein the collagen polypeptide comprises an amino acid sequence which is SEQ ID NO: 2.
19. The collagen polypeptide of any one of items 1-18, wherein the collagen polypeptide comprises an amino acid sequence which is at least 85% identical to any one of SEQ ID NOs: 3, 21 and 22.
20. The collagen polypeptide of any one of items 1-19, wherein the collagen polypeptide comprises an amino acid sequence which is selected from SEQ ID NOs: 3, 21 and 22.
21. The collagen polypeptide of any one of items 1-20, wherein the collagen polypeptide is a recombinant collagen polypeptide.
22. The collagen polypeptide of any one of items 1-21, wherein the collagen polypeptide is an isolated collagen polypeptide.
23. A collagen molecule comprising a homotrimer of three collagen polypeptides as defined in any one of items 1-22.
24. The collagen polypeptide of any one of items 1-22 or the collagen molecule of item 23, wherein the collagen polypeptide or the collagen molecule is glycosylated.
25. The collagen polypeptide or the collagen molecule of any one of the preceeding items, wherein the collagen polypeptide or the collagen molecule has been secreted from cultured yeast cells, preferably Pichia pastoris.
26. A nucleic acid encoding a collagen polypeptide as defined in any one of items 1-22 or 24-25.
27. A nucleic acid encoding a collagen polypeptide comprising an amino acid sequence of any one of SEQ ID NOs: 8, 10, 11, 12, 15 and 16, or a polypeptide which is at least 85% identical thereto.
28. The nucleic acid of item 27, wherein the encoded collagen polypeptide comprises the amino acid sequence of any one of SEQ ID NOs: 8, 10, 11, 12, 15 and 16.
29. An expression vector comprising the nucleic acid of any one of items 26-28.
30. A host cell comprising the expression vector of item 29 or the nucleic acid of any one of items 26-28.
31. A composition comprising the collagen polypeptide of any one of items 1-22 or 24-25, or the collagen molecule of any one of items 23-25, and one or more excipients.
32. The composition of item 31, wherein the composition is comprised in a cream, emulsion, serum, aqueous solution, ointment, paste, lotion, suspension, gel, mask, skin cleaner, cleansing cream, cleansing lotion, cleansing milk, cleansing pad, facial wash, hair shampoo, hair conditioner, or body shampoo.
33. The composition of item 31, wherein the composition is comprised in a tablet, capsule, granule or powder.
34. The composition of any one of items 31-33, wherein the composition comprises one or more additional active ingredients.
35. The composition of any one of items 31-34, wherein the composition is for topical use on a subject.
36. The composition of any one of items 31-35 for use in treating a skin disorder.
37. Use of the composition of any one of items 31-36 in repairing damaged skin, or protecting skin from oxidative damage, pigmentation or aging.
38. A method of repairing damaged skin, or protecting skin from oxidative damage, pigmentation or aging, comprising administering the composition of any one of items 31-36 to a subject.
39. The method item 38, wherein the composition is administered to the skin or skin cells of the subject.
40. Use of the composition of any one of items 31-36 as a skin moisturiser.
41. A method of preparing a recombinant collagen polypeptide or collagen molecule, comprising the following steps:
   (a) providing a nucleotide sequence encoding a collagen polypeptide comprising an amino acid sequence which is at least 85% identical to SEQ ID NO: 2, wherein the encoded collagen polypeptide has a molecular weight of 1-80 kDa;
   (b) optionally, providing a nucleotide sequence encoding a human prolyl-4-hydroxylase enzyme,
   (c) inserting the nucleotide sequences of (a) and optionally (b) into at least one vector, and
   (d) expressing the vector(s) of (c) in cultured yeast cells, preferably Pichia pastoris, and
   (e) isolating from the cultured yeast cells a recombinant collagen polypeptide or collagen molecule.
42. The method of item 41, wherein the encoded collagen polypeptide comprises an amino acid sequence which is SEQ ID NO: 2.
43. The method of item 41 or item 42, wherein the encoded collagen polypeptide has a molecular weight of 2 to 45 kDa.
44. The method of item 41 or item 42, wherein the encoded collagen polypeptide has a molecular weight of 5 to 20 kDa.
45. The method of any one of items 41-44, wherein the encoded collagen polypeptide comprises at least two PGP amino acid sequences.
46. The method of any one of items 41-45, wherein the amino acid sequence of the encoded collagen polypeptide comprises at least 2.5% hydroxyproline.
47. The method of any one of items 41-46, wherein the amino acid sequence of the encoded collagen polypeptide comprises at least 15% glycine.
48. The method of any one of items 41-47, wherein the encoded collagen polypeptide comprises hydroxylated lysines.
49. The method of any one of items 41-48, wherein the encoded collagen polypeptide has an isoelectric point of >7, preferably >8.
50. The method of any one of items 41-49, wherein the encoded collagen polypeptide is an antioxidant.
51. The method of any one of items 41-50, wherein the encoded collagen polypeptide has anti-aging activity.
52. The method of any one of items 41-51, wherein the encoded collagen polypeptide comprises an amino acid sequence which is GFSGLDGAKGD (SEQ ID NO: 6) or which differs from GFSGLDGAKGD (SEQ ID NO: 6) by up to three conservative amino acid substitutions.
53. The method of any one of items 41-52, wherein the encoded collagen polypeptide is hypoallergenic.
54. The method of any one of items 41-53, wherein the encoded collagen polypeptide comprises an amino acid sequence which is at least 85% identical to any one of SEQ ID NOs: 1 to 4 or 7 to 17.
55. The method of any one of items 41-53, wherein the encoded collagen polypeptide comprises an amino acid sequence which is selected from SEQ ID NOs: 1 to 4 and 7 to 17.
56. The method of any one of items 41-53, wherein the encoded collagen polypeptide comprises an amino acid sequence which is at least 85% identical to any one of SEQ ID NOs: 3, 21 and 22.
57. The method of any one of items 41-53, wherein the encoded collagen polypeptide comprises an amino acid sequence which is selected from any one of SEQ ID NOs: 3, 21 and 22.
58. The method of any one of items 41-57, wherein the recombinant collagen molecule comprises a homotrimer of three collagen polypeptides as defined in any one of items 1-22 and 24-28.
59. The method of any one of items 41-58, wherein the recombinant collagen polypeptide or collagen molecule is glycosylated.
60. The method of any one of items 41-59, wherein the nucleotide sequence encoding a collagen polypeptide comprising an amino acid sequence which is at least 85% identical to SEQ ID NO: 2 is preceded by a nucleotide sequence encoding a secretion signal sequence.
61. The method of item 60, wherein the nucleotide sequence encoding a secretion signal sequence encodes the amino acid sequence of SEQ ID NO: 5.
62. The method of any one of items 41-61, wherein the human prolyl-4-hydroxylase enzyme is encoded by amino acid sequences according to SEQ ID NO: 18 and SEQ ID NO: 19.
63. The method of any one of items 41-62, wherein the nucleotide sequences of (a) and/or (b) are inserted into a vector which uses a bi-directional promoter.
64. The method of any one of items 41-63, wherein the nucleotide sequences of (a) and/or (b) are inserted into a vector which uses an inducible and de-repressible promoter.
65. The method of item 64, wherein the promoter is methanol-inducible.
66. The method of any one of items 41-65, wherein the nucleotide sequences of (a) and/or (b) are inserted into a vector which allows post-translational translocation into the endoplasmic reticulum.
67. The method of any one of items 41-66, wherein the recombinant collagen polypeptide or collagen molecule is isolated from the culture supernatant of the cultured yeast cells.
68. A recombinant collagen polypeptide or collagen molecule prepared by the method of any one of items 41-68.
69. The recombinant collagen molecule of item 68, wherein the recombinant collagen molecule comprises a homotrimer of three collagen polypeptides.

### EXAMPLES

### Example 1: Development of Collagen Polypeptides and Collagen Molecules

Collagen amino acid sequences corresponding to fragments of human Type I A1 collagen (SEQ ID NO: 22) were used in the Examples (see, e.g., SEQ ID NO: 1 and SEQ ID NO: 2).

In order to balance water solubility and skin penetration, human Type I A1 collagen amino acid sequences with a molecular weight between 1 to 80 kDa, preferably 2 to 45 kDa, were selected.

Most proteins will reach their net charge state at a pH range of 5.5 to 8. This is the lowest solubility state of the protein called the isoelectric point. To facilitate the preparation of a skin care (typically pH 5.5) or personal care formulation, the protein should be in its soluble state, i.e., charged on its surface, at pH 5.5.

Amino acid sequences of human Type I A1 collagen were selected to contain prolines, and preferably to be enriched in prolines. The prolines in the amino acid sequences of collagen are hydroxylated in the endoplasmic reticulum during collagen synthesis. A higher hydroxyproline ratio in the collagen is expected to correspond to a higher moisturization and anti-aging activity. A higher hydroxyproline ratio is also expected to improve performance as a nutraceutical, as one potential mechanism of action involves the passage of hydroxyproline containing dipeptides and tripeptides into the the bloodstream.

The collagen polypeptides of the invention are based on a fragment of human Type I A1 collagen, which comprises the amino acid sequence of SEQ ID NO: 2 (see Tables below).

Some constructs for expression of collagen polypeptides were prepared with sequences encoding human Type I A1 collagen propeptide regions at the N-terminus (SEQ ID NO: 3) and/or the C-terminus (SEQ ID NO: 4), to further facilitate the formation of the collagen polypeptides into a higher order structure. For these constructs, KEX2 cleavage sites (“EKR”) were engineered between the collagen sequence and the C-terminal and N-terminal propeptide regions to enable cleaving off of C and/or N-terminal propeptides. KEX2 is a protease native to Pichia pastoris.

Some constructs were prepared with a nucleotide sequence encoding the yeast MatAlphaD secretion signal sequence (SEQ ID NO: 5) in order to facilitate secretion of the encoded collagen polypeptides from the Pichia pastoris cells. The MatAlphaD refers to a deletion variant of the mating factor alpha 1 prepro-peptide, which was shown to result in increased levels of some secreted proteins in comparison to the full-length signal sequence (G. P. Lin-Cereghino et al., Gene, 2013, 519, 311-317). MatAlphaD is believed to facilitate post-translational translocation into the endoplasmic reticulum (ER). Without wishing to be bound by theory, it is believed that a secretion signal sequence which increases residence time in the ER will increase the hydroxylation of the collagen polypeptide by co-expressed prolyl-4 hydroxylase in the ER. Controlling the period of time spent in the ER in this manner facilitates a balance between collagen hydroxylation and collagen polypeptide secretion.

The amino acid sequences referred to above are shown in Table 1.

TABLE 1
Amino acid sequences of polypeptides from human collagen
Type I A1 for expression and secretion in yeast

| | | Position in | |
|---|---|---|---|
| | | Human Type IA1 | |
| SEQ | Construct | Uniprot AA | |
| ID | Brief | sequence | |
| NO: | Title | (P02452) | FASTA Amino Acid Sequence |
| 1 | 400 AA | 541-940 | TGSPGSPGPDGKTGPPGPAGQDGRPGPPGPPGAR |
| | | | GQAGVMGFPGPKGAAGEPGKAGERGVPGPPGAVG |
| | | | PAGKDGEAGAQGPPGPAGPAGERGEQGPAGSPGF |
| | | | QGLPGPAGPPGEAGKPGEQGVPGDLGAPGPSGAR |
| | | | GERGFPGERGVQGPPGPAGPRGANGAPGNDGAKG |
| | | | DAGAPGAPGSQGAPGLQGMPGERGAAGLPGPKGD |
| | | | RGDAGPKGADGSPGKDGVRGLTGPIGPPGPAGAP |
| | | | GDKGESGPSGPAGPTGARGAPGDRGEPGPPGPAG |
| | | | FAGPPGADGQPGAKGEPGDAGAKGDAGPPGPAGP |
| | | | AGPPGPIGNVGAPGAKGARGSAGPPGATGFPGAA |
| | | | GRVGPPGPSGNAGPPGPPGPAGKEGGKGPRGETG |
| | | | PAGRPGEVGPPGPPGPAGEKGSPGAD |
| 2 | 80 AA | 220-300 | GPPGPPGKNGDDGEAGKPGRPGERGPPGPQGARG |
| | | | LPGTAGLPGMKGHRGFSGLDGAKGDAGPAGPKGE |
| | | | PGSPGENGAPGQ |
| 3 | N-terminal | 1-300 | MFSFVDLRLLLLLAATALLTHGQEEGQVEGQDED |
| | fragment | | IPPITCVQNGLRYHDRDVWKPEPCRICVCDNGKV |
| | (N) | | LCDDVICDETKNCPGAEVPEGECCPVCPDGSESP |
| | | | TDQETTGVEGPKGDTGPRGPRGPAGPPGRDGIPG |
| | | | QPGLPGPPGPPGPPGPPGLGGNFAPQLSYGYDEK |
| | | | STGGISVPGPMGPSGPRGLPGPPGAPGPQGFQGP |
| | | | PGEPGEPGASGPMGPRGPPGPPGKNGDDGEAGKP |
| | | | GRPGERGPPGPQGARGLPGTAGLPGMKGHRGFSG |
| | | | LDGAKGDAGPAGPKGEPGSPGENGAPGQ |
| 4 | C-terminal | 768-1464 | PIGPPGPAGAPGDKGESGPSGPAGPTGARGAPGD |
| | fragment | | RGEPGPPGPAGFAGPPGADGQPGAKGEPGDAGAK |
| | (C) | | GDAGPPGPAGPAGPPGPIGNVGAPGAKGARGSAG |
| | | | PPGATGFPGAAGRVGPPGPSGNAGPPGPPGPAGK |
| | | | EGGKGPRGETGPAGRPGEVGPPGPPGPAGEKGSP |
| | | | GADGPAGAPGTPGPQGIAGQRGVVGLPGQRGERG |
| | | | FPGLPGPSGEPGKQGPSGASGERGPPGPMGPPGL |
| | | | AGPPGESGREGAPGAEGSPGRDGSPGAKGDRGET |
| | | | GPAGPPGAPGAPGAPGPVGPAGKSGDRGETGPAG |
| | | | PTGPVGPVGARGPAGPQGPRGDKGETGEQGDRGI |
| | | | KGHRGFSGLQGPPGPPGSPGEQGPSGASGPAGPR |
| | | | GPPGSAGAPGKDGLNGLPGPIGPPGPRGRTGDAG |
| | | | PVGPPGPPGPPGPPGPPSAGFDFSFLPQPPQEKA |
| | | | HDGGRYYRADDANVVRDRDLEVDTTLKSLSQQIE |
| | | | NIRSPEGSRKNPARTCRDLKMCHSDWKSGEYWID |
| | | | PNQGCNLDAIKVFCNMETGETCVYPTQPSVAQKN |
| | | | WYISKNPKDKRHVWFGESMTDGFQFEYGGQGSDP |
| | | | ADVAIQLTFLRLMSTEASQNITYHCKNSVAYMDQ |
| | | | QTGNLKKALLLQGSNEIEIRAEGNSRFTYSVTVD |
| | | | GCTSHTGAWGKTVIEYKTTKTSRLPIIDVAPLDV |
| | | | GAPDQEFGFDVGPVCFL |
| | KEX2 | | EKR |
| | (protease) | | |
| 5 | MatAlphaD | | MRFPSIFTAVLFAASSALAAPVNTTTEDETAQIP |
| | (secretion | | AEAVIGYSDLEGDFDVAVLPFSASIAAKEEGVSL |
| | signal) | | EKREAEA |

As can be seen from the above, SEQ ID NOs: 1 to 4 contain PGP motifs.

Constructs for expression of collagen polypeptides, employing combinations of the sequences in Table 1, were designed and prepared. Some constructs were prepared for expression in yeast, such as Pichia pastoris.

Examples of these constructs are provided in Table 2, with the amino acid sequences they encode. SEQ ID NOs: 7-17 correspond to constructs #1-11, respectively.

The amino acid sequences of the collagen polypeptides produced with constructs #1-11 are indicated in bold and italics in Table 2.

TABLE 2
Constructs and their amino acid sequences

| | | | | | Calc. | |
|---|---|---|---|---|---|---|
| | | | | | mol. | |
| | | | | | weight | |
| | | | | | of | |
| | | | | | product | |
| | | | | | (after | |
| SEQ | | | | | cleavage | |
| ID NO: | N | C | | | and | |
| (con- | Pro- | Pro- | Other | | purifi- | |
| struct | peptide | peptide | modifi- | Signal | cation) | |
| #) | present | present | cations | Seq. | [kDa] | FASTA Amino Acid Sequence |
| 7 (#1) | No | No | — | Mat- | 35.1 | MRFPSIFTAVLFAASSALAAPVNTTTEDET |
| | | | | AlphaD | | AQIPAEAVIGYSDLEGDFDVAVLPFSASIA |
| | | | | | | AKEEGVSLEKREAEATGSPGSPGPDGKTGP |
| | | | | | | PGPAGQDGRPGPPGPPGARGQAGVMGFPGP |
| | | | | | | KGAAGEPGKAGERGVPGPPGAVGPAGKDGE |
| | | | | | | AGAQGPPGPAGPAGERGEQGPAGSPGFQGL |
| | | | | | | PGPAGPPGEAGKPGEQGVPGDLGAPGPSGA |
| | | | | | | RGERGFPGERGVQGPPGPAGPRGANGAPGN |
| | | | | | | DGAKGDAGAPGAPGSQGAPGLQGMPGERGA |
| | | | | | | AGLPGPKGDRGDAGPKGADGSPGKDGVRGL |
| | | | | | | TGPIGPPGPAGAPGDKGESGPSGPAGPTGA |
| | | | | | | RGAPGDRGEPGPPGPAGFAGPPGADGQPGA |
| | | | | | | KGEPGDAGAKGDAGPPGPAGPAGPPGPIGN |
| | | | | | | VGAPGAKGARGSAGPPGATGFPGAAGRVGP |
| | | | | | | PGPSGNAGPPGPPGPAGKEGGKGPRGETGP |
| | | | | | | AGRPGEVGPPGPPGPAGEKGSPGAD |
| 8 (#2) | No | No | — | Mat- | 7.3 | MRFPSIFTAVLFAASSALAAPVNTTTEDET |
| | | | | AlphaD | | AQIPAEAVIGYSDLEGDFDVAVLPFSASIA |
| | | | | | | AKEEGVSLEKREAEAGPPGPPGKNGDDGEA |
| | | | | | | GKPGRPGERGPPGPQGARGLPGTAGLPGMK |
| | | | | | | GHRGFSGLDGAKGDAGPAGPKGEPGSPGEN |
| | | | | | | GAPGQ |
| 9 (#3) | Yes | Yes | KEX2 | Mat- | 35.1 | MRFPSIFTAVLFAASSALAAPVNTTTEDET |
| | | | site | AlphaD | | AQIPAEAVIGYSDLEGDFDVAVLPFSASIA |
| | | | before | | | AKEEGVSLEKREAEAQEEGQVEGQDEDIPP |
| | | | and | | | ITCVQNGLRYHDRDVWKPEPCRICVCDNGK |
| | | | after | | | VLCDDVICDETKNCPGAEVPEGECCPVCPD |
| | | | terminal | | | GSESPTDQETTGVEGPKGDTGPRGPRGPAG |
| | | | fragments | | | PPGRDGIPGQPGLPGPPGPPGPPGPPGLGG |
| | | | | | | NFAPEKRTGSPGSPGPDGKTGPPGPAGQDG |
| | | | | | | RPGPPGPPGARGQAGVMGFPGPKGAAGEPG |
| | | | | | | KAGERGVPGPPGAVGPAGKDGEAGAQGPPG |
| | | | | | | PAGPAGERGEQGPAGSPGFQGLPGPAGPPG |
| | | | | | | EAGKPGEQGVPGDLGAPGPSGARGERGFPG |
| | | | | | | ERGVQGPPGPAGPRGANGAPGNDGAKGDAG |
| | | | | | | APGAPGSQGAPGLQGMPGERGAAGLPGPKG |
| | | | | | | DRGDAGPKGADGSPGKDGVRGLTGPIGPPG |
| | | | | | | PAGAPGDKGESGPSGPAGPTGARGAPGDRG |
| | | | | | | EPGPPGPAGFAGPPGADGQPGAKGEPGDAG |
| | | | | | | AKGDAGPPGPAGPAGPPGPIGNVGAPGAKG |
| | | | | | | ARGSAGPPGATGFPGAAGRVGPPGPSGNAG |
| | | | | | | PPGPPGPAGKEGGKGPRGETGPAGRPGEVG |
| | | | | | | PPGPPGPAGEKGSPGADEKRDDANVVRDRD |
| | | | | | | LEVDTTLKSLSQQIENIRSPEGSRKNPART |
| | | | | | | CRDLKMCHSDWKSGEYWIDPNQGCNLDAIK |
| | | | | | | VFCNMETGETCVYPTQPSVAQKNWYISKNP |
| | | | | | | KDKRHVWFGESMTDGFQFEYGGQGSDPADV |
| | | | | | | AIQLTFLRLMSTEASQNITYHCKNSVAYMD |
| | | | | | | QQTGNLKKALLLQGSNEIEIRAEGNSRFTY |
| | | | | | | SVTVDGCTSHTGAWGKTVIEYKTTKTSRLP |
| | | | | | | IIDVAPLDVGAPDQEFGFDVGPVCFL |
| 10 (#4) | Yes | Yes | KEX2 | Mat- | 7.3 | MRFPSIFTAVLFAASSALAAPVNTTTEDET |
| | | | site | AlphaD | | AQIPAEAVIGYSDLEGDFDVAVLPFSASIA |
| | | | before | | | AKEEGVSLEKREAEAQEEGQVEGQDEDIPP |
| | | | and | | | ITCVQNGLRYHDRDVWKPEPCRICVCDNGK |
| | | | after | | | VLCDDVICDETKNCPGAEVPEGECCPVCPD |
| | | | terminal | | | GSESPTDQETTGVEGPKGDTGPRGPRGPAG |
| | | | fragments | | | PPGRDGIPGQPGLPGPPGPPGPPGPPGLGG |
| | | | | | | NFAPEKRGPPGPPGKNGDDGEAGKPGRPGE |
| | | | | | | RGPPGPQGARGLPGTAGLPGMKGHRGFSGL |
| | | | | | | DGAKGDAGPAGPKGEPGSPGENGAPGQEKR |
| | | | | | | DDANVVRDRDLEVDTTLKSLSQQIENIRSP |
| | | | | | | EGSRKNPARTCRDLKMCHSDWKSGEYWIDP |
| | | | | | | NQGCNLDAIKVFCNMETGETCVYPTQPSVA |
| | | | | | | QKNWYISKNPKDKRHVWFGESMTDGFQFEY |
| | | | | | | GGQGSDPADVAIQLTFLRLMSTEASQNITY |
| | | | | | | HCKNSVAYMDQQTGNLKKALLLQGSNEIEI |
| | | | | | | RAEGNSRFTYSVTVDGCTSHTGAWGKTVIE |
| | | | | | | YKTTKTSRLPIIDVAPLDVGAPDQEFGFDV |
| | | | | | | GPVCFL |
| 11 (#5) | Yes | No | — | Mat- | 27.1 | MRFPSIFTAVLFAASSALAAPVNTTTEDET |
| | | | | AlphaD | | AQIPAEAVIGYSDLEGDFDVAVLPFSASIA |
| | | | | | | AKEEGVSLEKREAEAQEEGQVEGQDEDIPP |
| | | | | | | ITCVQNGLRYHDRDVWKPEPCRICVCDNGK |
| | | | | | | VLCDDVICDETKNCPGAEVPEGECCPVCPD |
| | | | | | | GSESPTDQETTGVEGPKGDTGPRGPRGPAG |
| | | | | | | PPGRDGIPGQPGLPGPPGPPGPPGPPGLGG |
| | | | | | | NFAPQLSYGYDEKSTGGISVPGPMGPSGPR |
| | | | | | | GLPGPPGAPGPQGFQGPPGEPGEPGASGPM |
| | | | | | | GPRGPPGPPGKNGDDGEAGKPGRPGERGPP |
| | | | | | | GPQGARGLPGTAGLPGMKGHRGFSGLDGAK |
| | | | | | | GDAGPAGPKGEPGSPGENGAPGQ |
| 12 (#6) | Yes | No | KEX2 | Mat- | 11.2 | MRFPSIFTAVLFAASSALAAPVNTTTEDET |
| | | | site | AlphaD | | AQIPAEAVIGYSDLEGDFDVAVLPFSASIA |
| | | | after | | | AKEEGVSLEKREAEAQEEGQVEGQDEDIPP |
| | | | terminal | | | ITCVQNGLRYHDRDVWKPEPCRICVCDNGK |
| | | | fragment | | | VLCDDVICDETKNCPGAEVPEGECCPVCPD |
| | | | | | | GSESPTDQETTGVEGPKGDTGPRGPRGPAG |
| | | | | | | PPGRDGIPGQPGLPGPPGPPGPPGPPGLGG |
| | | | | | | NFAPQLSYGYDEKSTGGISVPEKRGPMGPS |
| | | | | | | GPRGLPGPPGAPGPQGFQGPPGEPGEPGAS |
| | | | | | | GPMGPRGPPGPPGKNGDDGEAGKPGRPGER |
| | | | | | | GPPGPQGARGLPGTAGLPGMKGHRGFSGLD |
| | | | | | | GAKGDAGPAGPKGEPGSPGENGAPGQ |
| 13 (#7) | No | Yes | — | Mat- | 68.0 | MRFPSIFTAVLFAASSALAAPVNTTTEDET |
| | | | | AlphaD | | AQIPAEAVIGYSDLEGDFDVAVLPFSASIA |
| | | | | | | AKEEGVSLEKREAEAPIGPPGPAGAPGDKG |
| | | | | | | ESGPSGPAGPTGARGAPGDRGEPGPPGPAG |
| | | | | | | FAGPPGADGQPGAKGEPGDAGAKGDAGPPG |
| | | | | | | PAGPAGPPGPIGNVGAPGAKGARGSAGPPG |
| | | | | | | ATGFPGAAGRVGPPGPSGNAGPPGPPGPAG |
| | | | | | | KEGGKGPRGETGPAGRPGEVGPPGPPGPAG |
| | | | | | | EKGSPGADGPAGAPGTPGPQGIAGQRGVVG |
| | | | | | | LPGQRGERGFPGLPGPSGEPGKQGPSGASG |
| | | | | | | ERGPPGPMGPPGLAGPPGESGREGAPGAEG |
| | | | | | | SPGRDGSPGAKGDRGETGPAGPPGAPGAPG |
| | | | | | | APGPVGPAGKSGDRGETGPAGPTGPVGPVG |
| | | | | | | ARGPAGPQGPRGDKGETGEQGDRGIKGHRG |
| | | | | | | FSGLQGPPGPPGSPGEQGPSGASGPAGPRG |
| | | | | | | PPGSAGAPGKDGLNGLPGPIGPPGPRGRTG |
| | | | | | | DAGPVGPPGPPGPPGPPGPPSAGFDFSFLP |
| | | | | | | QPPQEKAHDGGRYYRADDANVVRDRDLEVD |
| | | | | | | TTLKSLSQQIENIRSPEGSRKNPARTCRDL |
| | | | | | | KMCHSDWKSGEYWIDPNQGCNLDAIKVFCN |
| | | | | | | METGETCVYPTQPSVAQKNWYISKNPKDKR |
| | | | | | | HVWFGESMTDGFQFEYGGQGSDPADVAIQL |
| | | | | | | TFLRLMSTEASQNITYHCKNSVAYMDQQTG |
| | | | | | | NLKKALLLQGSNEIEIRAEGNSRFTYSVTV |
| | | | | | | DGCTSHTGAWGKTVIEYKTTKTSRLPIIDV |
| | | | | | | APLDVGAPDQEFGFDVGPVCFL |
| 14 (#8) | No | Yes | KEX2 | Mat- | 40.4 | MRFPSIFTAVLFAASSALAAPVNTTTEDET |
| | | | site | AlphaD | | AQIPAEAVIGYSDLEGDFDVAVLPFSASIA |
| | | | before | | | AKEEGVSLEKREAEAPIGPPGPAGAPGDKG |
| | | | terminal | | | ESGPSGPAGPTGARGAPGDRGEPGPPGPAG |
| | | | fragment | | | FAGPPGADGQPGAKGEPGDAGAKGDAGPPG |
| | | | | | | PAGPAGPPGPIGNVGAPGAKGARGSAGPPG |
| | | | | | | ATGFPGAAGRVGPPGPSGNAGPPGPPGPAG |
| | | | | | | KEGGKGPRGETGPAGRPGEVGPPGPPGPAG |
| | | | | | | EKGSPGADGPAGAPGTPGPQGIAGQRGVVG |
| | | | | | | LPGQRGERGFPGLPGPSGEPGKQGPSGASG |
| | | | | | | ERGPPGPMGPPGLAGPPGESGREGAPGAEG |
| | | | | | | SPGRDGSPGAKGDRGETGPAGPPGAPGAPG |
| | | | | | | APGPVGPAGKSGDRGETGPAGPTGPVGPVG |
| | | | | | | ARGPAGPQGPRGDKGETGEQGDRGIKGHRG |
| | | | | | | FSGLQGPPGPPGSPGEQGPSGASGPAGPRG |
| | | | | | | PPGSAGAPGKDGLNGLPGPIGPPGPRGRTG |
| | | | | | | DAGPVGPPGPPGPPGPPGPPSAGFDFSFLP |
| | | | | | | QPPQEKAHDGGRYYRAEKRDDANVVRDRDL |
| | | | | | | EVDTTLKSLSQQIENIRSPEGSRKNPARTC |
| | | | | | | RDLKMCHSDWKSGEYWIDPNQGCNLDAIKV |
| | | | | | | FCNMETGETCVYPTQPSVAQKNWYISKNPK |
| | | | | | | DKRHVWFGESMTDGFQFEYGGQGSDPADVA |
| | | | | | | IQLTFLRLMSTEASQNITYHCKNSVAYMDQ |
| | | | | | | QTGNLKKALLLQGSNEIEIRAEGNSRFTYS |
| | | | | | | VTVDGCTSHTGAWGKTVIEYKTTKTSRLPI |
| | | | | | | IDVAPLDVGAPDQEFGFDVGPVCFL |
| 15 (#9) | Yes | Yes | KEX2 | Native | 7.3 | MFSFVDLRLLLLLAATALLTHGQEEGQVEG |
| | | | site | human | | QDEDIPPITCVQNGLRYHDRDVWKPEPCRI |
| | | | before | | | CVCDNGKVLCDDVICDETKNCPGAEVPEGE |
| | | | and | | | CCPVCPDGSESPTDQETTGVEGPKGDTGPR |
| | | | after | | | GPRGPAGPPGRDGIPGQPGLPGPPGPPGPP |
| | | | terminal | | | GPPGLGGNFAPEKRGPPGPPGKNGDDGEAG |
| | | | fragments | | | KPGRPGERGPPGPQGARGLPGTAGLPGMKG |
| | | | | | | HRGFSGLDGAKGDAGPAGPKGEPGSPGENG |
| | | | | | | APGQEKRDDANVVRDRDLEVDTTLKSLSQQ |
| | | | | | | IENIRSPEGSRKNPARTCRDLKMCHSDWKS |
| | | | | | | GEYWIDPNQGCNLDAIKVFCNMETGETCVY |
| | | | | | | PTQPSVAQKNWYISKNPKDKRHVWFGESMT |
| | | | | | | DGFQFEYGGQGSDPADVAIQLTFLRLMSTE |
| | | | | | | ASQNITYHCKNSVAYMDQQTGNLKKALLLQ |
| | | | | | | GSNEIEIRAEGNSRFTYSVTVDGCTSHTGA |
| | | | | | | WGKTVIEYKTTKTSRLPIIDVAPLDVGAPD |
| | | | | | | QEFGFDVGPVCFL |
| 16 (#10) | Yes | No | — | Native | 27.1 | MFSFVDLRLLLLLAATALLTHGQEEGQVEG |
| | | | | human | | QDEDIPPITCVQNGLRYHDRDVWKPEPCRI |
| | | | | | | CVCDNGKVLCDDVICDETKNCPGAEVPEGE |
| | | | | | | CCPVCPDGSESPTDQETTGVEGPKGDTGPR |
| | | | | | | GPRGPAGPPGRDGIPGQPGLPGPPGPPGPP |
| | | | | | | GPPGLGGNFAPQLSYGYDEKSTGGISVPGP |
| | | | | | | MGPSGPRGLPGPPGAPGPQGFQGPPGEPGE |
| | | | | | | PGASGPMGPRGPPGPPGKNGDDGEAGKPGR |
| | | | | | | PGERGPPGPQGARGLPGTAGLPGMKGHRGF |
| | | | | | | SGLDGAKGDAGPAGPKGEPGSPGENGAPGQ |
| 17 (#11) | No | Yes | — | Native | 68 | MFSFVDLRLLLLLAATALLTHGPIGPPGPA |
| | | | | human | | GAPGDKGESGPSGPAGPTGARGAPGDRGEP |
| | | | | | | GPPGPAGFAGPPGADGQPGAKGEPGDAGAK |
| | | | | | | GDAGPPGPAGPAGPPGPIGNVGAPGAKGAR |
| | | | | | | GSAGPPGATGFPGAAGRVGPPGPSGNAGPP |
| | | | | | | GPPGPAGKEGGKGPRGETGPAGRPGEVGPP |
| | | | | | | GPPGPAGEKGSPGADGPAGAPGTPGPQGIA |
| | | | | | | GQRGVVGLPGQRGERGFPGLPGPSGEPGKQ |
| | | | | | | GPSGASGERGPPGPMGPPGLAGPPGESGRE |
| | | | | | | GAPGAEGSPGRDGSPGAKGDRGETGPAGPP |
| | | | | | | GAPGAPGAPGPVGPAGKSGDRGETGPAGPT |
| | | | | | | GPVGPVGARGPAGPQGPRGDKGETGEQGDR |
| | | | | | | GIKGHRGFSGLQGPPGPPGSPGEQGPSGAS |
| | | | | | | GPAGPRGPPGSAGAPGKDGLNGLPGPIGPP |
| | | | | | | GPRGRTGDAGPVGPPGPPGPPGPPGPPSAG |
| | | | | | | FDFSFLPQPPQEKAHDGGRYYRADDANVVR |
| | | | | | | DRDLEVDTTLKSLSQQIENIRSPEGSRKNP |
| | | | | | | ARTCRDLKMCHSDWKSGEYWIDPNQGCNLD |
| | | | | | | AIKVFCNMETGETCVYPTQPSVAQKNWYIS |
| | | | | | | KNPKDKRHVWFGESMTDGFQFEYGGQGSDP |
| | | | | | | ADVAIQLTFLRLMSTEASQNITYHCKNSVA |
| | | | | | | YMDQQTGNLKKALLLQGSNEIEIRAEGNSR |
| | | | | | | FTYSVTVDGCTSHTGAWGKTVIEYKTTKTS |
| | | | | | | RLPIIDVAPLDVGAPDQEFGFDVGPVCFL |

In the tables above: Underlined sequence=MatAlphaD secretory signal (SEQ ID NO: 5); bold and italic sequences: sequences of collagen polypeptides produced with the respective construct.

The promoter used in the constructs was a methanol inducible promoter, which is also a de-repressible promoter meaning that its activity is already started at low concentration of the carbon source (e.g. glucose) which can then be boosted further by methanol addition. Accordingly, the promoter allows recombinant protein production under methanol-free conditions, if desired. And since there is already some transcription activity before the methanol induction, the cell can already adapt to this condition before expression is boosted.

As can be derived from Table 2, above, collagen polypeptides produced with constructs #2, #4, #5, #6, #9 and #10 (corresponding to SEQ ID NOs: 8, 10, 11, 12, 15 and 16, respectively) comprise the “80 AA” fragment of human Type I A1 collagen (SEQ ID NO: 2).

The sequences of the collagen polypeptides produced with constructs #2, #4, #5, #6, #9 and #10 are specified in Table 3, below.

The collagen polypeptides obtained with constructs #2, #4 and #9 correspond to the amino acid sequence of SEQ ID NO: 2. The collagen polypeptides obtained with constructs #5, #6 and #10 correspond to the amino acid sequences of SEQ ID NOs: 21, 22 and 3, respectively, which comprise SEQ ID NO: 2.

TABLE 3
Amino acid sequences of collagen polypeptides

| | FASTA Amino Acid Sequence of | SEQ ID |
|---|---|---|
| Construct # | collagen polypeptide | NO: |
| #2, #4, #9 | GPPGPPGKNGDDGEAGKPGRPGERGPPGPQGARGLPGTAGL | 2 |
| | PGMKGHRGFSGLDGAKGDAGPAGPKGEPGSPGENGAPGQ | |
| #5 | QEEGQVEGQDEDIPPITCVQNGLRYHDRDVWKPEPCRICVC | 21 |
| | DNGKVLCDDVICDETKNCPGAEVPEGECCPVCPDGSESPTD | |
| | QETTGVEGPKGDTGPRGPRGPAGPPGRDGIPGQPGLPGPPG | |
| | PPGPPGPPGLGGNFAPQLSYGYDEKSTGGISVPGPMGPSGP | |
| | RGLPGPPGAPGPQGFQGPPGEPGEPGASGPMGPRGPPGPPG | |
| | KNGDDGEAGKPGRPGERGPPGPQGARGLPGTAGLPGMKGHR | |
| | GFSGLDGAKGDAGPAGPKGEPGSPGENGAPGQ | |
| #6 | GPMGPSGPRGLPGPPGAPGPQGFQGPPGEPGEPGASGPMGP | 22 |
| | RGPPGPPGKNGDDGEAGKPGRPGERGPPGPQGARGLPGTAG | |
| | LPGMKGHRGFSGLDGAKGDAGPAGPKGEPGSPGENGAPGQ | |
| #10 | MFSFVDLRLLLLLAATALLTHGQEEGQVEGQDEDIPPITCV | 3 |
| | QNGLRYHDRDVWKPEPCRICVCDNGKVLCDDVICDETKNCP | |
| | GAEVPEGECCPVCPDGSESPTDQETTGVEGPKGDTGPRGPR | |
| | GPAGPPGRDGIPGQPGLPGPPGPPGPPGPPGLGGNFAPQLS | |
| | YGYDEKSTGGISVPGPMGPSGPRGLPGPPGAPGPQGFQGPP | |
| | GEPGEPGASGPMGPRGPPGPPGKNGDDGEAGKPGRPGERGP | |
| | PGPQGARGLPGTAGLPGMKGHRGFSGLDGAKGDAGPAGPKG | |
| | EPGSPGENGAPGQ | |

The amino acid sequences of human prolyl-4-hydroxylase (P4HA & P4HB) and human collagen Type I A1 are provided in Table 4, below.

TABLE 4
Amino acid sequences of human prolyl-4-hydroxylase
(P4HA & P4HB) and human collagen Type I A1

| SEQ | | |
|---|---|---|
| ID | | |
| NO: | Subunit | FASTA Amino Acid Sequence |
| 18 | P4Ha | MIWYILIIGILLPQSLAHPGFFTSIGQMTDLIHTEKDLVTSLKDYIKAEEDKL |
| | | EQIKKWAEKLDRLTSTATKDPEGFVGHPVNAFKLMKRLNTEWSELENLVLKDM |
| | | SDGFISNLTIQRQYFPNDEDQVGAAKALLRLQDTYNLDTDTISKGNLPGVKHK |
| | | SFLTAEDCFELGKVAYTEADYYHTELWMEQALRQLDEGEISTIDKVSVLDYLS |
| | | YAVYQQGDLDKALLLTKKLLELDPEHQRANGNLKYFEYIMAKEKDVNKSASDD |
| | | QSDQKTTPKKKGVAVDYLPERQKYEMLCRGEGIKMTPRRQKKLFCRYHDGNRN |
| | | PKFILAPAKQEDEWDKPRIIRFHDIISDAEIEIVKDLAKPRLRRATISNPITG |
| | | DLETVHYRISKSAWLSGYENPVVSRINMRIQDLTGLDVSTAEELQVANYGVGG |
| | | QYEPHFDFARKDEPDAFKELGTGNRIATWLFYMSDVSAGGATVFPEVGASVWP |
| | | KKGTAVFWYNLFASGEGDYSTRHAACPVLVGNKWVSNKWLHERGQEFRRPCTL |
| | | SELE |
| 19 | P4Hb | DAPEEEDHVLVLRKSNFAEALAAHKYLLVEFYAPWCGHCKALAPEYAKAAGKL |
| | | KAEGSEIRLAKVDATEESDLAQQYGVRGYPTIKFFRNGDTASPKEYTAGREAD |
| | | DIVNWLKKRTGPAATTLPDGAAAESLVESSEVAVIGFFKDVESDSAKQFLQAA |
| | | EAIDDIPFGITSNSDVFSKYQLDKDGVVLFKKFDEGRNNFEGEVTKENLLDFI |
| | | KHNQLPLVIEFTEQTAPKIFGGEIKTHILLFLPKSVSDYDGKLSNFKTAAESF |
| | | KGKILFIFIDSDHTDNQRILEFFGLKKEECPAVRLITLEEEMTKYKPESEELT |
| | | AERITEFCHRFLEGKIKPHLMSQELPEDWDKQPVKVLVGKNFEDVAFDEKKNV |
| | | FVEFYAPWCGHCKQLAPIWDKLGETYKDHENIVIAKMDSTANEVEAVKVHSFP |
| | | TLKFFPASADRTVIDYNGERTLDGFKKFLESGGQDGAGDDDDLEDLEEAEEPD |
| | | MEEDDDQKAVKDEL |
| 20 | Human | MFSFVDLRLLLLLAATALLTHGQEEGQVEGQDEDIPPITCVQNGLRYHDRDVW |
| | Type\|A1 | KPEPCRICVCDNGKVLCDDVICDETKNCPGAEVPEGECCPVCPDGSESPTDQE |
| | Uniprot | TTGVEGPKGDTGPRGPRGPAGPPGRDGIPGQPGLPGPPGPPGPPGPPGLGGNF |
| | AA | APQLSYGYDEKSTGGISVPGPMGPSGPRGLPGPPGAPGPQGFQGPPGEPGEPG |
| | sequence | ASGPMGPRGPPGPPGKNGDDGEAGKPGRPGERGPPGPQGARGLPGTAGLPGMK |
| | (P02452) | GHRGFSGLDGAKGDAGPAGPKGEPGSPGENGAPGQMGPRGLPGERGRPGAPGP |
| | | AGARGNDGATGAAGPPGPTGPAGPPGFPGAVGAKGEAGPQGPRGSEGPQGVRG |
| | | EPGPPGPAGAAGPAGNPGADGQPGAKGANGAPGIAGAPGFPGARGPSGPQGPG |
| | | GPPGPKGNSGEPGAPGSKGDTGAKGEPGPVGVQGPPGPAGEEGKRGARGEPGP |
| | | TGLPGPPGERGGPGSRGFPGADGVAGPKGPAGERGSPGPAGPKGSPGEAGRPG |
| | | EAGLPGAKGLTGSPGSPGPDGKTGPPGPAGQDGRPGPPGPPGARGQAGVMGFP |
| | | GPKGAAGEPGKAGERGVPGPPGAVGPAGKDGEAGAQGPPGPAGPAGERGEQGP |
| | | AGSPGFQGLPGPAGPPGEAGKPGEQGVPGDLGAPGPSGARGERGFPGERGVQG |
| | | PPGPAGPRGANGAPGNDGAKGDAGAPGAPGSQGAPGLQGMPGERGAAGLPGPK |
| | | GDRGDAGPKGADGSPGKDGVRGLTGPIGPPGPAGAPGDKGESGPSGPAGPTGA |
| | | RGAPGDRGEPGPPGPAGFAGPPGADGQPGAKGEPGDAGAKGDAGPPGPAGPAG |
| | | PPGPIGNVGAPGAKGARGSAGPPGATGFPGAAGRVGPPGPSGNAGPPGPPGPA |
| | | GKEGGKGPRGETGPAGRPGEVGPPGPPGPAGEKGSPGADGPAGAPGTPGPQGI |
| | | AGQRGVVGLPGQRGERGFPGLPGPSGEPGKQGPSGASGERGPPGPMGPPGLAG |
| | | PPGESGREGAPGAEGSPGRDGSPGAKGDRGETGPAGPPGAPGAPGAPGPVGPA |
| | | GKSGDRGETGPAGPTGPVGPVGARGPAGPQGPRGDKGETGEQGDRGIKGHRGF |
| | | SGLQGPPGPPGSPGEQGPSGASGPAGPRGPPGSAGAPGKDGLNGLPGPIGPPG |
| | | PRGRTGDAGPVGPPGPPGPPGPPGPPSAGFDFSFLPQPPQEKAHDGGRYYRAD |
| | | DANVVRDRDLEVDTTLKSLSQQIENIRSPEGSRKNPARTCRDLKMCHSDWKSG |
| | | EYWIDPNQGCNLDAIKVFCNMETGETCVYPTQPSVAQKNWYISKNPKDKRHVW |
| | | FGESMTDGFQFEYGGQGSDPADVAIQLTFLRLMSTEASQNITYHCKNSVAYMD |
| | | QQTGNLKKALLLQGSNEIEIRAEGNSRFTYSVTVDGCTSHTGAWGKTVIEYKT |
| | | TKTSRLPIIDVAPLDVGAPDQEFGFDVGPVCFL |

### Example 2: Protein Secretion Screening and Characterisation of a Fragment of Col1a1 Collagen From Pichia Pastoris

We overexpressed constructs encoding human Type I A1 collagen polypeptides according to the constructs defined in Example 1.

We co-overexpressed the machinery for hydroxylation of prolines in the Pichia pastoris system (prolyl-4-hydroxylase (P4HA & B heterotetramer A2B2, from Homo sapiens) under a bidirectional promoter by random integration of the construct in the genome

### Materials and Methods

Pichia pastoris Transformation

P. pastoris P4H platform strain was transformed with 1 μg of Smil linearized expression constructs by electroporation. After 3 h of regeneration in 1 mL YPD/Sorbitol (1:1) at 30° C. and 550 rpm cells were plated on YPD agar plates containing 100 mg/L Zeocin.

Pichia pastoris Cultivation and Induction of Expression

### Deep-Well Plate Cultivation Protocols:

Methanol-depended deep-well plate cultivation protocol (screening and re-screening):
Prepare medium in 96 well deep well plates under sterile conditions. Inoculate 250 μL BMG1 with a fresh single colony of the target P. pastoris strain using sterile tooth picks.
Culture for 60 hours at 28° C., 320 rpm under 80% humidity.
After 60 hours of growth save the clones by stamping them to YPD agar square plates and induce cultures by addition of 250 μL BMM2.
Further induction by addition of 50 μL BMM10 after 68, 84 and 92 hours after inoculation Stop cultivation after a total of 108 hours (=48 hours of induction).
Centrifuge cells at 4000 rpm, 4° C. for 10 min, harvest supernatant and apply activity assay or run an SDS-PAGE analysis to detect the target protein.

Derepressed (methanol-free) deep-well plate cultivation protocol (screening):
Prepare medium in 96 well deep well plates under sterile conditions.
Inoculate 250 μL BMG1 with a fresh single colony of the target P. pastoris strain using sterile tooth picks.
Culture for 60 hours at 28° C., 320 rpm under 80% humidity.
After 60 hours of growth save the clones by stamping them to YPD agar square plates and induce cultures by addition of 250 μL BMG0.5.
Further induction by addition of 50 μL BMG2.5 after 68, 84 and 92 hours after inoculation Stop cultivation after a total of 108 hours (=48 hours of induction).
Centrifuge cells at 4000 rpm, 4° C. for 10 min, harvest supernatant and apply activity assay or run an SDS-PAGE or native gel analysis to detect the target protein.

### Reagents, Chemicals and Material Composition

sterile 250 mL to 2.5 L baffled shake flasks.
YPD (1% w/v yeast extract, 2% w/v peptone, 2% w/v glucose).
BMM10 (buffered minimal methanol containing 5% methanol: 1.34% yeast nitrogen base w/o amino acids, 4×10-5% biotin, 200 mM potassium phosphate buffer, pH 6.0 and 5% methanol).
BMM2 (buffered minimal methanol containing 5% methanol: 1.34% yeast nitrogen base w/o amino acids, 4×10-5% biotin, 200 mM potassium phosphate buffer, pH 6.0 and 1% methanol).
BMG1 (buffered minimal methanol containing 5% methanol: 1.34% yeast nitrogen base w/o amino acids, 4×10-5% biotin, 200 mM potassium phosphate buffer, pH 6.0 and 1% glycerol).
BMG0.5 (buffered minimal methanol containing 5% methanol: 1.34% yeast nitrogen base w/o amino acids, 4×10-5% biotin, 200 mM potassium phosphate buffer, pH 6.0 and 0.5% glycerol).
BMG2.5 (buffered minimal methanol containing 5% methanol: 1.34% yeast nitrogen base w/o amino acids, 4×10-5% biotin, 200 mM potassium phosphate buffer, pH 6.0 and 2.5% glycerol).
Pure Methanol.

### SDS Page Protocol:

Protein analysis by electrophoresis in denaturing gels (SDS-PAGE): To assess the protein production profile by separating the proteins based on their molecular weight. The standard protocol for running Bolt Bis-Tris Plus Mini Gels:
Centrifuge broth (4000 rcf, 10 min). Separate supernatant from cell fraction.
Measure protein concentration in each sample. Normalise the protein concentration in samples to analyse.
Mix sample with 4× loading buffer containing LDS, and 10× reducing agent containing DTT. Complete with distilled water to reach 30 μL.
Load 15-20 μL of sample/well in a BisTris 4-16% gel.
Run in MES running buffer (200 V, 25 min)
Resolve gel by incubating 45 min in BlueSafe protein stain and washing in distilled water overnight.
   SDS PAGE with Pepsin Digested Protein Suspension:
The protein solution was diluted in a 50 mM potassium phosphate buffer (pH 2.2) after lyophilization and treated with 0.05 mg/mL pepsin at 22° C., 20 h.

### Results

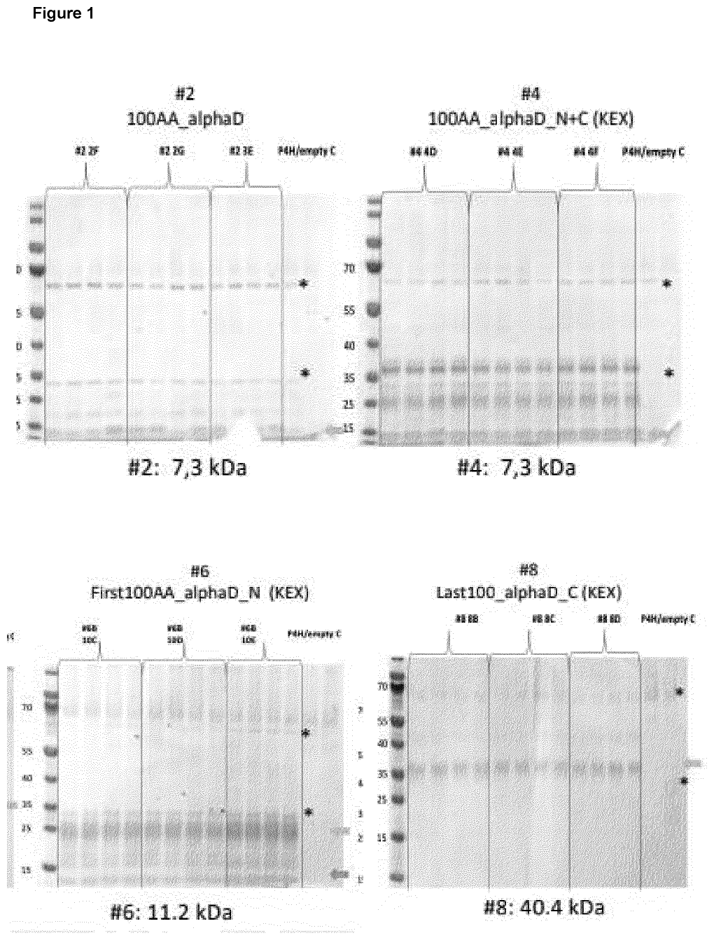

The fragments of overexpressed collagen in Pichia pastoris are shown by the SDS-PAGE gels in FIG. 1. These SDS PAGE gels show the expression of collagen polypeptides for constructs #2, 4, 6, and 8 (construct #2 (SEQ ID NO: 8), 4 (SEQ ID NO: 10), 6 (SEQ ID NO: 12), and 8 (SEQ ID NO: 14)—see Table 2). The stars show the two subunits of the P4H machinery.

### Characterisation of Homotrimer Formation of the COL1A1 Fragments of Human Collagen in Pichia Pastoris

According to Vuorela et al. EMBO Journal 1997, Vol. 16 No. 22, there is a complicated regulation between overexpression of the P4H machinery and assembled collagen fragments. The P4HA subunit was previously described as being retained in the ER. In our experiments, it was occasionally secreted into the supernatant—see FIG. 1, which shows for example that when co-expressed with construct #2 (SEQ ID NO: 8) the P4H subunits were secreted into the supernatant.

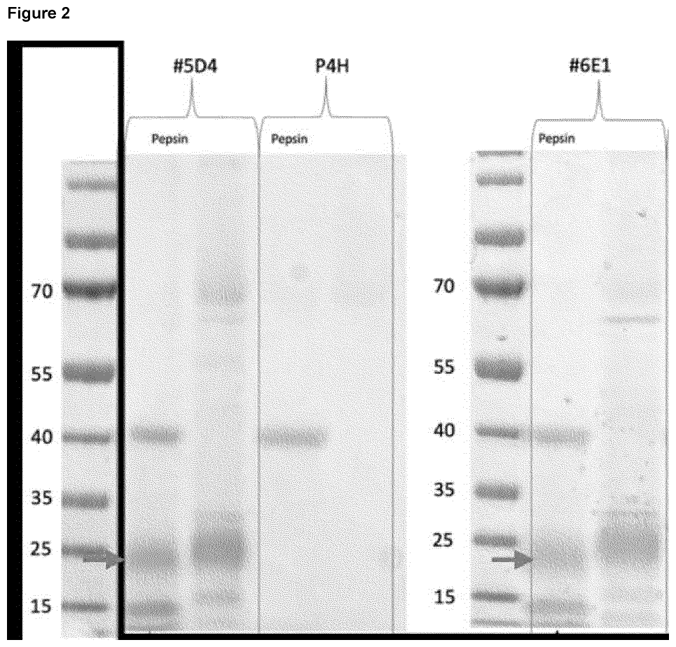

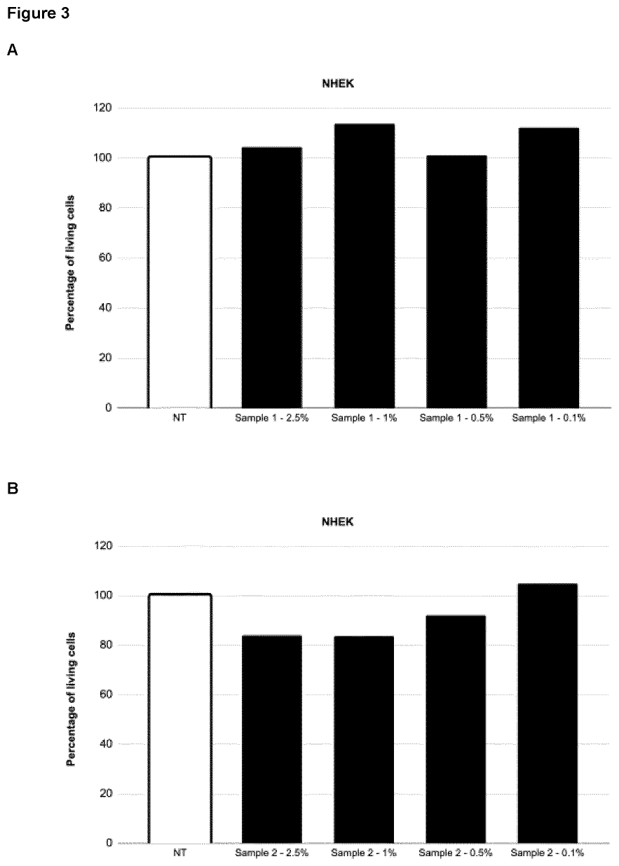

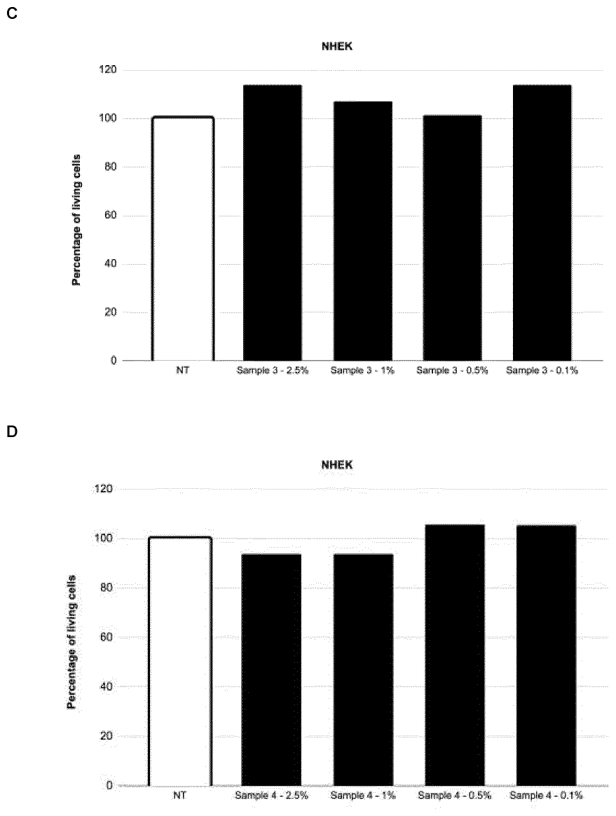

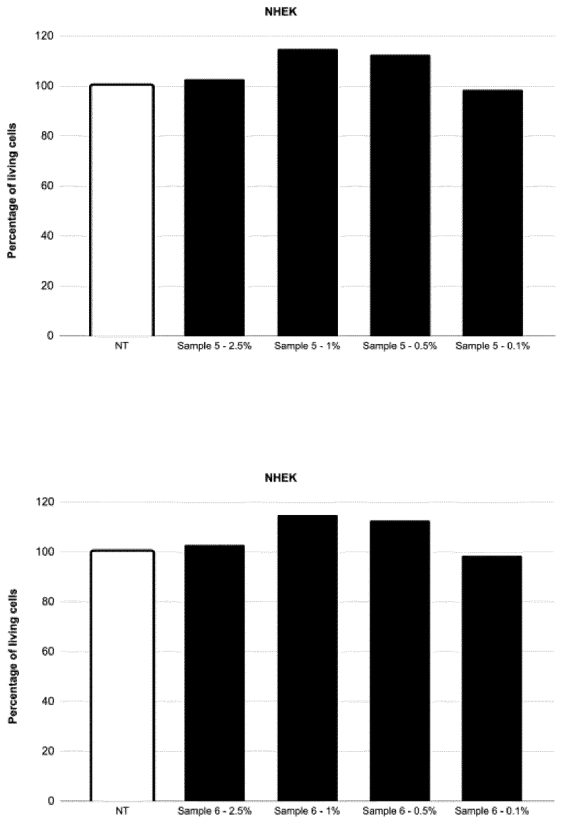

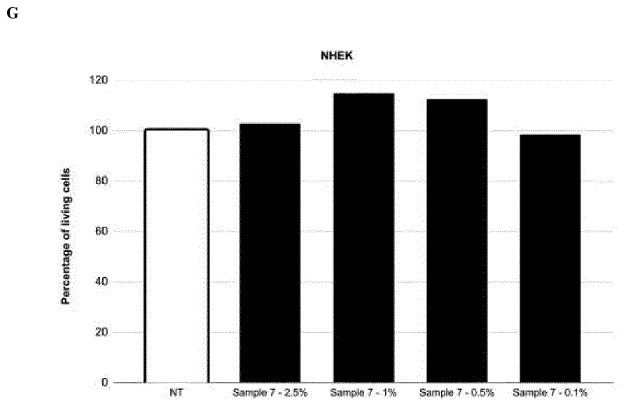

Further SDS-PAGE analysis in FIG. 2 shows homotrimeric collagen fragments produced and secreted by Pichia pastoris, as the full-length constructs which migrate at around ˜25 kDa (for constructs #5 and 6 (SEQ ID NO: 11 and SEQ ID NO: 12, respectively, indicated by red arrows) as well as any cleaved products (e.g. SEQ ID NO: 21 and 22) are shown to be resistant to digestion by pepsin, which is the canonical way to show homotrimeric structure.

### Example 3: Amino Acid Sequencing

### Method

Analysis of the produced peptides by NanoHPLC-MS/MS.
Identification of proteins found in the secretome of selected strains.
Analysis of proline hydroxylation as post translational modification (PTM).
Coverage of at least 50% is expected.
Proteolytic enzyme: trypsin suggested as standard.

### Results

The peptide analysis results from two isolated bands from a SDS gel are shown below:
   Collagen polypeptide 1 (SEQ ID NO: 2): 7.3 kDa band, generated with construct #2 (see FIG. 2)
   Collagen polypeptide 2 (SEQ ID NO: 22): 11.2 kDa band, generated with construct #6 (see FIG. 2)
Proteolytic in-gel digestion (Trypsin) and analysis of the resulting peptides on a tandem mass spectrometer revealed that all selected protein bands appear to be collagen.
Peptides belonging to the collagen in question were found amongst the 10 most abundant peptides in the analyzed samples.
For all constructs analyzed, processing of the secretion peptide is presumed to be complete, since none of the samples showed peptides corresponding to the secretion signal MatAlphaD.
   The following results are a deep dive into the bands referred to above:
Collagen polypeptide 1 (SEQ ID NO: 2)
   Sequence coverage of the collagen is 55%.
   Analysis revealed at least 28% of native sites for proline hydroxylation were hydroxylated in the peptide, corresponding to 2.5% hydroxyproline.
Collagen polypeptide 2 (SEQ ID NO: 22)
   Sequence coverage of the collagen is 43%.
   The collagen fragment and the N-Terminal and the C-Terminal fragments were found.
Analysis revealed at least 15% of native sites for proline hydroxylation were hydroxylated in the peptide, corresponding to 1.6% hydroxyproline.

### Example 4: Cytotoxicity Assay and Gene Expression Analysis

Changes in expression of different genes described to be implicated in skin aging were determined by mRNA sequencing in normal human dermal keratinocytes (NHEKs) upon treatment with different collagens.

### Methods

### 1. Cytotoxicity Assay

### Test Model:

First, cytotoxicity of different products (Samples 1-7, below) at different concentrations on normal human dermal keratinocytes (NHEKs) was determined.

### Samples:

The products tested are labelled as follows:
SAMPLE 1—a buffer solution (PBS) to re-suspend the target protein
SAMPLE 2—the target protein (SEQ ID NO: 2) at 2.25 w % in a buffer solution (see SAMPLE 1)—Cambrium vegan human collagen
SAMPLE 3—a buffer solution (20 mM acetic acid) to re-suspend the bovine collagen and the hydrolyzed collagen
SAMPLE 4—bovine collagen at 2.25 w % in a buffer solution (see SAMPLE 3)—Gibco™ Collagen 1, bovine (commercially available from ThermoFisher; product code A1064401; Nov. 23, 2018)
SAMPLE 5—hydrolysed collagen at 2.25 w % in a buffer solution (see SAMPLE 3)—iCONFIT hydrolysed collagen (obtained from iCONFIT European Foods 00, Tallinn)
SAMPLE 6—a buffer solution to re-suspend the synthetic collagen (composed of Aqua, hexanediol 5 w/v %, Butylene glycol 5 w/v %)
SAMPLE 7—A synthetic collagen solution in a buffer solution (see SAMPLE 6)—INCI: sh-polypeptide-121

### Protocol:

All samples were diluted directly in appropriate culture medium at 4 different concentrations: 2.5%, 1%, 0.5% and 0.1% (dilution factors 40, 100, 200 and 1000). The experiment was carried out in triplicates (n=3).

The products were incubated with NHEKs for 24 hours at 37° C. During the last 3 hours of the 24-hour incubation period, cell proliferation reagent WST1 (Roche) was introduced into the culture medium. WST1 contains tetrazolium salts which are cleaved into a formazan dye (a yellow indicator) by metabolically active cells. The level of yellow staining (determined by the absorbance at 450 nm) is proportional to the number of living cells and a value between 80% and 120% of the untreated control indicates an absence of cytotoxicity of the product.

### 2. Gene Expression Analysis

### Test Model:

Gene expression changes in normal human dermal keratinocytes (NHEKs) upon treatment with Samples 1-7 were determined by mRNA sequencing.

### Samples:

The experiment was carried out in triplicates (n=3) with the combinations shown below:
NHEKs untreated (NT)
NHEKs+Sample 1 at 2.5%
NHEKs+Sample 2 at 2.5%
NHEKs+Sample 3 at 2.5%
NHEKs+Sample 4 at 2.5%
NHEKs+Sample 5 at 2.5%
NHEKs+Sample 6 at 2.5%
NHEKs+Sample 7 at 2.5%

In detail, NHEKs were cultured at a concentration of 10,000 cells per well in a 96-well plate in the presence of suitable culture media (Promocell). After adhesion, Samples 1-7 at 2.5% (see above) were added to the NHEKs and incubated for 24 hours.

Subsequently, mRNA was extracted from the NHEKs, subjected to reverse transcription and DNA was prepared for sequencing according to the Fluidigm protocol (96 genes) used for real-time PCR.

The samples were tested on a list of genes known to be involved in skin cellular pathways related to common cosmetic claims.

### Results

### 1. Cytotoxicity Assay

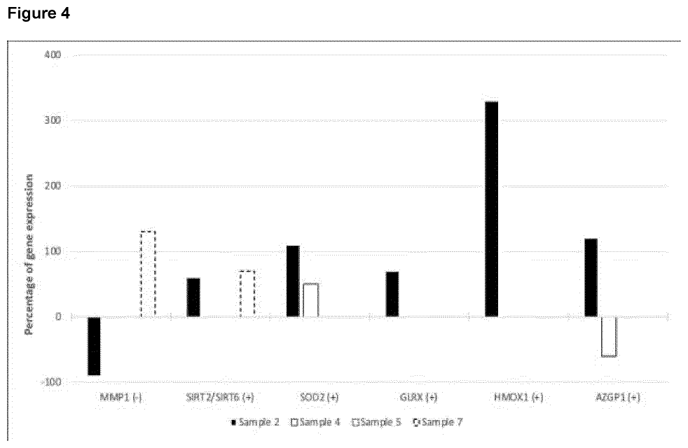

No cytotoxic activity on NHEKs was detected for Samples 1-7 at a concentration of 2.5% or below (as indicated by a percentage of living cells of 80-120% of the untreated control cells; see FIG. 4).

### 2. Gene Expression Analysis

Table 5 and FIG. 5 show the changes in the expression of specific genes associated with curative and preventive anti-aging and other beneficial skin effects in NHEKs upon treatment with Samples 2, 4, 5 and 7 (indicated is the percentage of up-/down-regulation in relation to the basal level in NHEKs).

Only changes greater than 50% induction and less than 50% inhibition are shown.

TABLE 5
Biological activity of Samples 2, 4, 5 and 7 on the expression of different genes in NHEKs

| Described effect | Genes names | Sample 2 | Sample 4 | Sample 5 | Sample 7 |
|---|---|---|---|---|---|
| Curative Anti-aging (ECM | MMP1 (−) | −90% | no | no | +130% |
| protection) | | | activity | activity | |
| Preventive Anti-aging (Survival | SIRT2/SIRT6 (+) | +60% | no | no | +70% |
| growth and renewal) | | | activity | activity | |
| Preventive Anti-aging | SOD2 (+) | +110 | +50 | no | no |
| (antioxidant) | | | | activity | activity |
| Preventive Anti-aging | GLRX (+) | +70 | no | no | no |
| (antioxidant) | | | activity | activity | activity |
| Preventive Anti-aging | HMOX1 (+) | +330 | no | no | no |
| (antioxidant) | | | activity | activity | activity |
| Pigmentation | AZGP1 (+) | +120 | −60% | no | no |
| (Antipigmentation) | | | | activity | activity |

In the Table above: MMP1: Matrix metallopeptidase 1; SIRT2/SIRT6: Sirtuin 2 and 6; SOD2: Superoxide dismutase 2 mitochondrial; GLRX: Glutaredoxin; HMOX1: Heme oxygenase 1; AZGP1: Alpha-2-glycoprotein 1, zinc-binding; (−) indicates that the under-expression of this gene may be associated with beneficial effects on the skin. (+) indicates that the over-expression of this gene may be associated with beneficial activity for the skin.

### Discussion

Remarkably, treatment with Sample 2 affected expression of a variety of different genes (up- and down-regulation) described to be involved in preventive and curative anti-aging, antioxidant and anti-pigmentation in NHEK cells, as compared to treatment with other collagens (see Table 5: inhibition of expression of MMP1; activation of expression of SIRT2/SIRT6, SOD2, GLRX, HMOX1 and AZGP1).

Up- and down-regulation of the expression of these genes in NHEKs upon treatment with Sample 2 suggests beneficial effects on skin as summarized in Table 6, below.

TABLE 6
Overview about differentially expressed genes in NHEKs upon treatment with Sample 2

| | | | | Expected | |
|---|---|---|---|---|---|
| | | | | activity for | |
| | Official | Described | | beneficial | |
| Gene | Name | Activity | Mode of action | effect | Literature |
| MMP1 | Matrix | Extracellular | Enzyme capable of degrading | Inhibition | (1) Shin S. Y., |
| | metallopeptidase | Matrix - | collagens. | (−) | Molecular Biology |
| | 1 | Degradation | Enzyme responsible for the first | | Reports, 2021 |
| | | (curative anti- | step in the degradation of | | January; 48(1): 1-11 |
| | | aging) | collagens. Inhibition is also | | |
| | | | described to be associated with | | |
| | | | improved wound healing | | |
| | | | outcomes. | | |
| SIRT2/ | Sirtuin 2 and | Cell | Sirtuin 2 regulates cell | Activation | (2) Ming M. et al., |
| SIRT6 | 6 | protection | proliferation and promotes cell | (+) | Experimental |
| | | and longevity | longevity. | | Dermatoloy, 2014 |
| | | (preventive | Sirtuin 6 is involved in the repair | | March; 23(3): 207-9 |
| | | anti-aging) | of double-strand breaks in DNA | | |
| | | | and preserving telomeres. Its | | |
| | | | overexpression protects cells | | |
| | | | from senescence. | | |
| SOD2 | Superoxide | Antioxidant | Superoxide dismutase 2 is a | Activation | (3) Dunaway S. et |
| | dismutase 2 | (preventive | mitochondrial enzyme with | (+) | al., Frontiers in |
| | | anti-aging) | antioxidant activity. It is also | | Pharmacology, |
| | | | known to reduce UV-induced | | 2018 April 24; 9: 392 |
| | | | pigmentation. | | (4) Shin S. et al., |
| GRLX | Glutaredoxin | Antioxidant | Glutaredoxin is a cytoplasmic | Activation | Journal of Cosmetic |
| | | (preventive | enzyme catalyzing the reversible | (+) | Dermatology, 2020 |
| | | anti-aging) | reduction of glutathione-protein | | April; 19(4): 977-984 |
| | | | mixed disulfides. This enzyme | | |
| | | | highly contributes to the | | |
| | | | antioxidant defense system. It | | |
| | | | protects mitochondria maintaining | | |
| | | | the membrane potential. | | |
| HMOX1 | Heme | Antioxidant | Heme oxygenase is involved in | Activation | |
| | oxygenase 1 | (preventive | the resistance to environmental | (+) | |
| | | anti-aging) | stress. It acts as a cytoprotective | | |
| | | | enzyme. | | |
| AZGP1 | Alpha-2- | Anti- | Natural inhibitor of melanogenesis | Activation | |
| | glycoprotein | pigmentation | produced by keratinocytes | (+) | |
| | 1, zinc- | | | | |
| | binding | | | | |

The generated data thus indicate that treatment with Sample 2 was associated with beneficial skin activity in preventative and in curating aging by activating the antioxidant defences of the skin as an effective way to prevent aging, as well as by inhibiting the breakdown of collagen and contributing to the regulation of cell proliferation and longevity as an effective way to modulate several effects due to aging.

### CONCLUSION

These data suggest that the collagen polypeptides of the present invention exhibit strong anti-aging activity by affecting transcriptional regulation of a variety of different genes implicated in skin aging thereby slowing down intrinsic aging processes in skin cells.

1.-22. (canceled)

23. A collagen polypeptide comprising an amino acid sequence which is at least 85% identical to SEQ ID NO: 2, wherein the collagen polypeptide has a molecular weight of 1 to 80 kDa.

24. The collagen polypeptide of claim 23, wherein the collagen polypeptide comprises the amino acid sequence of SEQ ID NO: 2.

25. The collagen polypeptide of claim 23, wherein the amino acid sequence of the collagen polypeptide comprises at least 2.5% hydroxyproline.

26. The collagen polypeptide of claim 23, wherein the collagen polypeptide has an isoelectric point of >7.

27. The collagen polypeptide of claim 23, wherein the collagen polypeptide has an isoelectric point of >8.

28. The collagen polypeptide of claim 23, wherein the collagen polypeptide has a molecular weight of 2 to 45 kDa.

29. The collagen polypeptide of claim 28, wherein the collagen polypeptide has a molecular weight of 5 to 20 kDa.

30. The collagen polypeptide of claim 23, wherein the collagen polypeptide has anti-aging activity.

31. A collagen molecule comprising a homotrimer of three collagen polypeptides as defined in claim 23.

32. A nucleic acid encoding a collagen polypeptide as defined in claim 23.

33. An expression vector comprising the nucleic acid of claim 32.

34. A host cell comprising the expression vector of claim 33 or the nucleic acid of claim 32.

35. A composition comprising the collagen polypeptide of claim 23 or the collagen molecule of claim 31, and one or more excipients.

36. The composition of claim 35, wherein the composition is comprised in a cream, emulsion, serum, aqueous solution, ointment, paste, lotion, suspension, gel, mask, skin cleaner, cleansing cream, cleansing lotion, cleansing milk, cleansing pad, facial wash, hair shampoo, hair conditioner, or body shampoo.

37. The composition of claim 35, wherein the composition is comprised in a tablet, capsule, granule or powder.

38. The composition of claim 35, wherein the composition comprises one or more additional active ingredients.

39. A method of preparing a recombinant collagen polypeptide or collagen molecule, comprising the following steps:
(a) providing a nucleotide sequence encoding a collagen polypeptide comprising an amino acid sequence which is at least 85% identical to SEQ ID NO: 2, wherein the encoded collagen polypeptide has a molecular weight of 1-80 kDa;
(b) optionally, providing a nucleotide sequence encoding a human prolyl-4-hydroxylase enzyme,
(c) inserting the nucleotide sequences of (a) and optionally (b) into at least one vector, and
(d) expressing the vector(s) of (c) in cultured yeast cells, and
(e) isolating from the cultured yeast cells a recombinant collagen polypeptide or collagen molecule.

40. The method of claim 39, wherein the yeast cells are Pichia pastoris cells.

41. The method of claim 39, wherein the nucleotide sequences of (a) and/or (b) are inserted into a vector which allows post-translational translocation into the endoplasmic reticulum.

42. The method of claim 39, wherein the recombinant collagen polypeptide or collagen molecule is isolated from the culture supernatant of the cultured yeast cells.

43. A recombinant collagen polypeptide or collagen molecule prepared by the method of claim 39.
